SEQUENCE LISTING (36 sequences)

SEQ ID NO: 1 - protein, 5 aa, synthetic construct
DYNVH

SEQ ID NO: 2 - protein, 17 aa, synthetic construct
YFYPRNGATHYNQKFTG

SEQ ID NO: 3 - protein, 17 aa, synthetic construct
YFYPRNGATHYAQGFTG

SEQ ID NO: 4 - protein, 17 aa, synthetic construct
YFYPRNGATHYAQKFQG

SEQ ID NO: 5 - protein, 17 aa, synthetic construct
YFYPRNGATHYAEKFQG

SEQ ID NO: 6 - protein, 9 aa, synthetic construct
GETPGTFPY

SEQ ID NO: 7 - protein, 15 aa, synthetic construct
RASESVDNFGITFMH

SEQ ID NO: 8 - protein, 15 aa, synthetic construct
QASESVDNFGITFMH

SEQ ID NO: 9 - protein, 7 aa, synthetic construct
RASNLES

SEQ ID NO: 10 - protein, 7 aa, synthetic construct
RASNLET

SEQ ID NO: 11 - protein, 9 aa, synthetic construct
QQSSKDPRT

SEQ ID NO: 12 - protein, 118 aa, synthetic construct
QVQLVQSGSELKKPGASVKVSCKASGYTFTDYNVHWIRQAPGQGLEWIGYFYPRNGATHYNQKFTGRAVLSADTSVSTAYLQISSLKAEDTAVYFCARGETPGTFPYWGQGTLVTVSS

SEQ ID NO: 13 - protein, 118 aa, synthetic construct
QVQLVQSGSELKKPGASVKVSCKASGYTFTDYNVHWIRQAPGQGLEWIGYFYPRNGATHYAQGFTGRAVLSADTSVSTAYLQISSLKAEDTAVYYCARGETPGTFPYWGQGTLVTVSS

SEQ ID NO: 14 - protein, 118 aa, synthetic construct
QVQLVQSGAEVKKPGASVKVSCKASGYTFTDYNVHWIRQAPGQGLEWIGYFYPRNGATHYNQKFTGRATLTVDTSTSTAYMEISSLRSEDTAVYFCARGETPGTFPYWGQGTLVTVSS

SEQ ID NO: 15 - protein, 118 aa, synthetic construct
QVQLVQSGAEVKKPGASVKVSCKASGYTFTDYNVHWIRQAPGQGLEWIGYFYPRNGATHYAQKFQGRVTMTVDTSTSTAYMELSSLRSEDTAVYYCARGETPGTFPYWGQGTLVTVSS

SEQ ID NO: 16 - protein, 118 aa, synthetic construct
EVQLVQSGAEVKKPGATVKISCKASGYTFTDYNVHWIQQAPGKGLEWIGYFYPRNGATHYNQKFTGRATLTADTSTDTAYMEISSLRSEDTAVYFCARGETPGTFPYWGQGTLVTVSS

SEQ ID NO: 17 - protein, 118 aa, synthetic construct
EVQLVQSGAEVKKPGATVKISCKASGYTFTDYNVHWIQQAPGKGLEWIGYFYPRNGATHYAEKFQGRVTLTADTSTDTAYMELSSLRSEDTAVYYCARGETPGTFPYWGQGTLVTVSS

SEQ ID NO: 18 - protein, 111 aa, synthetic construct
DIVLTQSPASLAVSPGQRATITCRASESVDNFGITFMHWYQQKPGQPPKLLIYRASNLESGVPARFSGSGSRTDFTLTINPVEANDTANYYCQQSSKDPRTFGQGTKLEIK

SEQ ID NO: 19 - protein, 111 aa, synthetic construct
EIVLTQSPATLSLSPGERATLSCRASESVDNFGITFMHWYQQKPGQAPRLLIYRASNLESGIPARFSGSGSRTDFTLTISSLEPEDFAVYYCQQSSKDPRTFGQGTKLEIK

SEQ ID NO: 20 - protein, 111 aa, synthetic construct
DIQLTQSPSSLSASVGDRVTITCRASESVDNFGITFMHWYQQKPGKAPKLLIYRASNLESGVPSRFSGSGSRTDFTLTISSLQPEDFATYYCQQSSKDPRTFGQGTKLEIK

SEQ ID NO: 21 - protein, 111 aa, synthetic construct
DIQLTQSPSSLSASVGDRVTITCQASESVDNFGITFMHWYQQKPGKAPKLLIYRASNLETGVPSRFSGSGSRTDFTFTISSLQPEDIATYYCQQSSKDPRTFGQGTKLEIK

SEQ ID NO: 22 - protein, 285 aa, synthetic construct
  REGION 1..19: note = single peptide
  REGION 278..285: note = GS linker and His tag
MKHLWFFLLLVAAPRWVLSVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEIGSHHHHHH

SEQ ID NO: 23 - protein, 118 aa, synthetic construct
EVQLQESGAELVRSGASVKMSCKASGYTFTDYNVHWIKQTPGQGLEWIGYFYPRNGATHYNQKFTGKATLTADTSSSTAYIQISSLTSEDSAVYFCARGETPGTFPYWGQGTLVTVSA

SEQ ID NO: 24 - protein, 111 aa, synthetic construct
DIVLTQSPASLTVSLGQRATISCRASESVDNFGITFMHWYQQKPGQPPKLLIYRASNLESGIPARFSGSGSRTDFTLTIDPVETDDVATYYCQQSSKDPRTFGGGTKLEIK

SEQ ID NO: 25 - protein, 330 aa, synthetic construct [sequence omitted]

SEQ ID NO: 26 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 225 aa, synthetic construct
DKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 28 - protein, 448 aa, synthetic construct
QVQLVQSGSELKKPGASVKVSCKASGYTFTDYNVHWIRQAPGQGLEWIGYFYPRNGATHYNQKFTGRAVLSADTSVSTAYLQISSLKAEDTAVYFCARGETPGTFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 448 aa, synthetic construct
QVQLVQSGAEVKKPGASVKVSCKASGYTFTDYNVHWIRQAPGQGLEWIGYFYPRNGATHYNQKFTGRATLTVDTSTSTAYMEISSLRSEDTAVYFCARGETPGTFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 446 aa, synthetic construct
QVQLVQSGSELKKPGASVKVSCKASGYTFTDYNVHWIRQAPGQGLEWIGYFYPRNGATHYNQKFTGRAVLSADTSVSTAYLQISSLKAEDTAVYFCARGETPGTFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 31 - protein, 446 aa, synthetic construct
QVQLVQSGAEVKKPGASVKVSCKASGYTFTDYNVHWIRQAPGQGLEWIGYFYPRNGATHYNQKFTGRATLTVDTSTSTAYMEISSLRSEDTAVYFCARGETPGTFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 32 - protein, 24 aa, synthetic construct
MDPKGSLSWRILLFLSLAFELSYG

SEQ ID NO: 33 - protein, 107 aa, synthetic construct [sequence omitted]

SEQ ID NO: 34 - protein, 218 aa, synthetic construct
DIVLTQSPASLAVSPGQRATITCRASESVDNFGITFMHWYQQKPGQPPKLLIYRASNLESGVPARFSGSGSRTDFTLTINPVEANDTANYYCQQSSKDPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 218 aa, synthetic construct
DIQLTQSPSSLSASVGDRVTITCQASESVDNFGITFMHWYQQKPGKAPKLLIYRASNLETGVPSRFSGSGSRTDFTFTISSLQPEDIATYYCQQSSKDPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 256 aa, Homo sapiens
RWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

(19) United States
(12) Patent Application Publication
(10) Pub. No.: US 2026/0217850 A1
(43) Pub. Date: Jul. 30, 2026

(54) CD38 MONOCLONAL ANTIBODY AND USE THEREOF

(71) Applicant: SOUND BIOPHARMACEUTICALS CO. LTD., Chengdu, Sichuan (CN)

(72) Inventors: Fanxin Ma, Chengdu, Sichuan (CN); Qing Li, Chengdu, Sichuan (CN); Ying Huang, Chengdu, Sichuan (CN); Xianda Zhang, Chengdu, Sichuan (CN); Pengyu Chen, Chengdu, Sichuan (CN); Zhouning Yang, Chengdu, Sichuan (CN); Shuang Liu; Feng Qu, Chengdu, Sichuan (CN); Chunmei Chen, Chengdu, Sichuan (CN)

(73) Assignee: SOUND BIOPHARMACEUTICALS CO. LTD., Chengdu, Sichuan (CN)

(21) Appl. No.: 19/140,989

(22) PCT Filed: Dec. 20, 2023
(86) PCT No.: PCT/CN2023/140300

§ 371 Date: Jun. 18, 2025

(30) Foreign Application Priority Data
Dec. 21, 2022 (CN) 202211652420.9

(51) Int. Cl. C07K 16/28; A61K 39/00; A61P 35/00

(52) U.S. Cl. CPC C07K 16/2896; A61P 35/00; A61K 2039/505; C07K 2317/24; C07K 2317/92

(57) ABSTRACT

Disclosed is a monoclonal antibody specifically binding to human CD38, or an antigen binding fragment thereof, wherein the monoclonal antibody binds to an extracellular domain of CD38 at amino acid residues comprising at least one amino acid residue selected from a group consisting of K25, E28, 129, M33, and R34, wherein the amino acid residue is numbered according to the amino acid sequence of the extracellular domain of CD38 as shown in SEQ ID NO: 36. Also disclosed is a pharmaceutical composition comprising the antibodies and medical uses thereof.

### TECHNICAL FIELD

The present disclosure relates to a monoclonal antibody binding to CD38 and particularly to a humanized monoclonal antibody against human CD38, an antigen-binding fragment thereof, a related pharmaceutical composition, and use thereof in treating disease.

### BACKGROUND

CD38 is a type II transmembrane glycoprotein that plays a role in the regulation of cell migration and receptor-mediated adhesion through interactions with CD31 or hyaluronic acid. CD38 also has extracellular enzymatic activity, is involved in the production of nucleotide metabolites, and plays a role in controlling intracellular calcium storage.

CD38 is commonly found on hematopoietic cells and is present in solid tissues at low levels. The expression of CD38 in hematopoietic cells depends on the differentiation and activation states of the cells. Under normal conditions, CD38 is expressed at relatively low levels in bone marrow and lymphocytes, as well as in some non-hematopoietic tissues. Approximately 80% of resting NK cells and monocytes express low levels of CD38, and so do various other blood cell types, including lymph node germinal center lymphoblasts, intrafollicular cells, dendritic cells, erythrocytes, and platelets. In contrast, CD38 is also expressed on B cells, and normal plasma cells express particularly high levels of CD38.

CD38 is expressed in numerous hematologic malignancies, such as Waldenström's macroglobulinemia, primary systemic amyloidosis, mantle cell lymphoma, acute lymphoblastic leukemia, acute myeloid leukemia, NK cell leukemia, NK/T cell lymphoma, plasma cell leukemia, chronic lymphocytic leukemia (CLL), and multiple myeloma (MM). CD38 is particularly expressed at high levels in multiple myeloma (MM) cells, making CD38 a target for therapeutic antibodies that target MM cell surface molecules.

In addition, there have been some reports that CD38 antibodies may also be therapeutic in other malignancies, such as small cell lung cancer, non-small cell lung cancer, lung bronchial epithelial cancer, breast cancer (evolving from malignant hyperplasia of the epithelial lining in the ducts and lobules of the breast), pancreatic tumor (insulinoma) evolving from β cells, tumors evolving from the epithelium in the intestine (such as adenocarcinoma and squamous cell carcinoma), prostate cancer, testicular seminoma, ovarian cancer, and neuroblastoma. Many studies have also suggested the role of CD38 in autoimmunity, such as in Graves' disease and thyroiditis, type 1 and type 2 diabetes, and inflammation of airway smooth muscle cells during asthma. In addition, CD38 expression is also associated with HIV infection.

Daratumumab (hereinafter referred to as “Dara” or “DARA” or “dara”) is the first CD38-targeting antibody, and it has been approved as a drug used alone or in combination with other drugs for treating multiple myeloma. Recently, the CD38 antibody isatuximab (hereinafter referred to as “Isa”) by Sanofi has also been approved by the FDA as a treatment for relapsed/refractory multiple myeloma (R/R MM). CD38 mAbs under clinical validation also include TJ202/MOR202 (I-MAB Biopharma/MorphoSys AG), SG301 (Sumgen), HLX15 (Henlius), and TAK-079 (Takeda). Thus, it is necessary to provide more CD38 antibodies to meet clinical needs.

### SUMMARY

The present invention provides a monoclonal antibody binding to human CD38 or an antigen-binding fragment thereof, comprising three heavy chain complementarity-determining regions HCDR1, HCDR2, and HCDR3, and three light chain complementarity-determining regions LCDR1, LCDR2, and LCDR3, wherein: the amino acid sequence of the HCDR1 is set forth in SEQ ID NO: 1 or a variant thereof with one to three conservative amino acid substitutions; the amino acid sequence of the HCDR2 is set forth in SEQ ID NO: 2 or a variant thereof with one to three conservative amino acid substitutions; the amino acid sequence of the HCDR3 is set forth in SEQ ID NO: 6 or a variant thereof with one to three conservative amino acid substitutions; the amino acid sequence of the LCDR1 is set forth in SEQ ID NO: 7 or a variant thereof with one to three conservative amino acid substitutions; the amino acid sequence of the LCDR2 is set forth in SEQ ID NO: 9 or a variant thereof with one to three conservative amino acid substitutions; and the amino acid sequence of the LCDR3 is set forth in SEQ ID NO: 11 or a variant thereof with one to three conservative amino acid substitutions.

In some embodiments, the monoclonal antibody or the antigen-binding fragment thereof comprises a heavy chain variable region VH and a light chain variable region VL, wherein the VH comprises the amino acid sequence set forth in SEQ ID NO: 12 or SEQ ID NO: 14 or a variant having at least 85% sequence identity thereto, and the VL comprises the amino acid sequence set forth in SEQ ID NO: 18 or SEQ ID NO: 21 or a variant having at least 85% sequence identity thereto.

In some embodiments, the monoclonal antibody or the antigen-binding fragment thereof comprises a heavy chain variable region VH and a light chain variable region VL, wherein (a) the heavy chain variable region VH comprises the amino acid sequence set forth in SEQ ID NO: 12 or a variant having at least 85% sequence identity thereto, and the light chain variable region VL comprises the amino acid sequence set forth in SEQ ID NO: 18 or a variant having at least 85% sequence identity thereto; or (b) the heavy chain variable region VH comprises the amino acid sequence set forth in SEQ ID NO: 12 or a variant having at least 85% sequence identity thereto, and the light chain variable region VL comprises the amino acid sequence set forth in SEQ ID NO: 21 or a variant having at least 85% sequence identity thereto; or (c) the heavy chain variable region VH comprises the amino acid sequence set forth in SEQ ID NO: 14 or a variant having at least 85% sequence identity thereto, and the light chain variable region VL comprises the amino acid sequence set forth in SEQ ID NO: 18 or a variant having at least 85% sequence identity thereto.

In some embodiments, an Fc portion of the monoclonal antibody is modified to enhance binding to FcγRIIIa (V) and/or FcγRIIIa (F). In some embodiments, the Fc portion comprises the amino acid sequence set forth in SEQ ID NO: 27 or a variant having at least 85% sequence identity thereto.

In some embodiments, the monoclonal antibody or the antigen-binding fragment thereof comprises a heavy chain and a light chain, wherein the heavy chain comprises the amino acid sequence set forth in SEQ ID NO: 28, SEQ ID NO: 29, SEQ ID NO: 30, or SEQ ID NO: 31 or a variant having at least 85% sequence identity thereto, and the light chain comprises the amino acid sequence set forth in SEQ ID NO: 34 or SEQ ID NO: 35 or a variant having at least 85% sequence identity thereto.

In some embodiments, the antigen-binding fragment is selected from an scFv, an (scFv) 2, a Fab, a Fab′, and a F(ab′)₂ of the CD38 antibody. In some embodiments, the antigen-binding fragment forms a portion of a multispecific antibody, CAR-T, or BiTE.

In some embodiments, the monoclonal antibody is of an IgG type. In some embodiments, the monoclonal antibody is of the IgG1, IgG2, IgG3, or IgG4 type. In some embodiments, the monoclonal antibody is of the IgG1 type. In some embodiments, the monoclonal antibody or the antigen-binding fragment thereof does not bind to healthy human erythrocytes. In some embodiments, the monoclonal antibody or the antigen-binding fragment thereof is capable of binding to healthy human PBMCs. In some embodiments, the monoclonal antibody or the antigen-binding fragment thereof has a 10⁻⁹ M level affinity constant Kd as determined by Biacore. Alternatively, the antigen-binding fragment thereof binds to a different antigenic epitope than isatuximab does. In some embodiments, the monoclonal antibody or the antigen-binding fragment thereof binds to a different antigenic epitope than daratumumab does.

In another aspect, the present disclosure provides a monoclonal antibody or an antigen-binding fragment thereof, which competes for binding to human CD38 with any of the monoclonal antibodies or antigen-binding fragments provided above by the present disclosure. In some embodiments, the present disclosure provides a monoclonal antibody or an antigen-binding fragment thereof, which competes for binding to human CD38 with FTL004-1, FTL004-6, or FTL004-7 provided by the present disclosure or an antigen-binding fragment thereof.

In another aspect, the present disclosure provides a monoclonal antibody specifically binding to human CD38, or an antigen-binding fragment thereof, which binds to an extracellular domain of CD38 at at least one amino acid residue selected from a group consisting of K25, E28, 129, M33, and R34, wherein the amino acid residue is numbered according to the extracellular domain of CD38 having an amino acid sequence shown in SEQ ID NO: 36. In some embodiments, the monoclonal antibody or an antigen-binding fragment thereof binds to the extracellular domain of CD38 at amino acid residues consisting of K25, E28, 129, P31, E32, M33, R34, H35, K67, T70, Q71, T72, and V73.

In another aspect, the present invention provides a pharmaceutical composition comprising the monoclonal antibody or the antigen-binding fragment thereof of the present disclosure, and a pharmaceutically acceptable carrier.

In some embodiments, the pharmaceutical composition further comprises a second therapeutic agent for treating the same cancer. In some embodiments, the second therapeutic agent is a chemotherapeutic agent, a radiotherapeutic agent, or a biological agent. In some embodiments, the biological agent is a monoclonal antibody, an ADC, an oncolytic virus, or CAR-T therapy.

In another aspect, the present disclosure also provides a nucleotide sequence encoding the monoclonal antibody or the antigen-binding fragment thereof of the present invention. In another aspect, the present disclosure also provides a vector comprising the aforementioned nucleotide sequence. In another aspect, the present disclosure also provides a non-human host cell comprising the aforementioned vector. In addition, the present disclosure also provides a cell line that produces the monoclonal antibody or the antigen-binding fragment thereof of the present disclosure, a recombinant expression vector comprising the nucleotide of the present disclosure, and a method for preparing an antibody by culturing an antibody-producing cell line.

In another aspect, the present disclosure also provides use of the monoclonal antibody or the antigen-binding fragment thereof or the pharmaceutical composition of the present invention in the preparation of a medicament for treating a CD38-positive cancer or an autoimmune disease.

In another aspect, the present invention also provides a method for treating a CD38-positive cancer or an autoimmune disease in a subject, comprising administering to the subject a therapeutically effective amount of any of the monoclonal antibodies or antigen-binding fragments thereof of the present invention or any of the pharmaceutical compositions of the present invention. In some embodiments, the method also comprises administering to the subject the second therapeutic agent simultaneously or in any order.

### BRIEF DESCRIPTION OF THE DRAWINGS

FIG. 1. The apoptotic activity of the H2 antibody on Daudi, Ramos, Raji, and SU-DHL-1 cells.

FIG. 2. Identification through an Elisa assay for the H2 antibody competing with Isa and Dara for binding to an antigenic epitope.

FIG. 3. Elisa assays for the cross-reactivity of the antibodies between CD38 antigen proteins of different species.

FIG. 4. Testing of the apoptotic activity of the humanized antibodies and the positive control on Ramos and Raji cells.

FIG. 5. ELISA assays for the activity of the humanized CD38 antibodies.

FIG. 6. Tumor growth curves for the humanized antibodies FTL004-hu-1/6/7.

FIG. 7. Tumor inhibition rates for the humanized antibodies FTL004-hu-1/6/7.

FIG. 8. The hepatotoxicity and nephrotoxicity of the humanized antibodies FTL004-hu-1/6/7.

FIG. 9. Elisa detection of changes in the activity of the humanized antibodies FTL004-1/6/7.

FIG. 10. Tumor growth curves for different concentrations of FTL004-1, FTL004-7, FTL004-hu-1, Isa, and FTL004-1+Dara.

FIG. 11. Tumor inhibition rates for different concentrations of FTL004-1, FTL004-7, FTL004-hu-1, Isa, and FTL004-1+Dara.

FIG. 12. Immunohistochemistry detection of CD38 expression in tumor tissues.

FIG. 13. The in vivo killing effect of the CD38 antibodies on small cell lung cancer H211 cells.

FIG. 14. Flow cytometry assays for the binding of the CD38 antibodies to H211 and SU-DHL-6 cells.

FIG. 15. Flow cytometry assays for the binding reactions of the CD38 antibodies with normal human erythrocytes.

FIG. 16. Flow cytometry assays for the binding reactions of the CD38 antibodies, Dara and the antibody FTL004-1, with normal human PBMCs.

FIG. 17. Three-Dimensional structure analysis of the complex formed by CD38 extracellular domain and FTL004-1 Fab (CD38/FTL004-1-Fab). (A) The general structure of the CD38/FTL004-1-Fab. CD38 and FTL004-1 heavy and light chains were colored grey, purple and green, respectively. (B) CDRs in the CD38/FTL004-1-Fab complex. (C) Amino acid sequence of CD38 extracellular domain (R45-1300), in which the residues of CD38 that bind to FTL004-1-Fab were colored blue. (D) Stereogram showing direct hydrogen bonds and salt bridges of the interaction between CD38 and FTL004-1, in which hydrogen bonds were shown in black broken line sand salt bridges were shown in purple broken lines. (E) Comparing the bindings of FTL004-1, daratumumab and isatuximab. Epitopes on CD38 (grey) were colored green (for FTL004-1), blue (for daratumumab), red (for isatuximab), and yellow (for the overlap of FTL004-1 and isatuximab). (F) Proposed direction of CD38 on cellular memberane and the binding epitopes for FTL004-1, daratumumab, and isatuximab. The transmembrane helix (dark golden) was proposed as its extended N terminus of CD38.

### DETAILED DESCRIPTION

### Definitions

As used herein, the term “patient” or “subject” refers to any organism to which the provided antibody, the antigen-binding fragment thereof, or the pharmaceutical composition is administered or can be administered for experimental, diagnostic, prophylactic, cosmetic, and/or therapeutic purposes. Typical subjects include animals (e.g., mammals such as mice, rats, rabbits, non-human primates, and/or humans). In some embodiments, the subject is a human. In some embodiments, the subject is suffering from or susceptible to one or more disorders or conditions. The patient may exhibit one or more symptoms of a disorder or condition, or may have been diagnosed with one or more disorders or conditions (e.g., cancers/tumors). In some embodiments, the patient is receiving or has received some therapy for diagnosing and/or treating such a disease, disorder, or condition.

As used herein, the term “comparable” means that two or more agents, entities, circumstances, effects, sets of conditions, etc. may not be identical to one another but are sufficiently similar to permit comparison (e.g., by level and/or activity) therebetween, so that conclusions may be reasonably drawn based on the differences or similarities observed. Such comparable sets of conditions, effects, circumstances, individuals, or populations are characterized by multiple substantially identical features and one or a small number of varied features. Those of ordinary skill in the art should understand, in context, what degree of identity is required in any given circumstance for two or more such agents, entities, circumstances, sets of conditions, effects, populations, or the like to be considered comparable.

A composition or method described herein as “comprising” one or more said elements or steps is open-ended, meaning that the elements or steps are essential, but other elements or steps may be added within the scope of the composition or method. It is also understood that any composition or method described as “comprising” one or more said elements or steps also describes the corresponding, more limited composition or method “consisting substantially of the elements or steps”, meaning that the composition or method includes the essential elements or steps and may also include additional elements or steps that do not substantially affect the basic and novel characteristics of the composition or method.

As used herein, the term “epitope” refers to a portion of an antigen to which an antibody or antigen-binding fragment binds. In some embodiments, the epitope may be a conformational epitope, i.e., portions of the antigen that are not covalently contiguous in the antigen but that are near to one another in three-dimensional space when the antigen is in a relevant conformation. For example, for CD38, conformational epitopes are those, in the extracellular domain of CD38, composed of amino acid residues that are not contiguous. In some embodiments, the epitope may be a linear epitope, i.e., an epitope comprising a sequence of amino acid residues contiguous in the primary structure in the extracellular domain of CD38. Means for determining the exact sequence and/or in particular amino acid residues of an epitope of CD38 are known in the literature, including competing with peptides from the antigen sequence for binding to CD38 sequences from different species, truncation and/or mutagenesis (e.g. by alanine scanning or other site-directed mutagenesis), phage display-based screening, or (co-) crystallography techniques. In the present disclosure, an amino acid residue of CD38 extracellular domain that is bound by an antibody of the present disclosure is defined by an amino acid residue that is within 5 Å away from any atom of the CDRs of the present antibody.

As used herein, an amino acid residue is designated as its single letter abbreviation plus its position in relation to a reference sequence. For example, in the sentence “binds to an extracellular domain of CD38 at amino acid residues comprising at least one amino acid residue selected from the group consisting of K25, E28, 129, M33. R34, and V73, wherein the amino acid residue is numbered according to the extracellular domain of CD38 having an amino acid sequence shown in SEQ ID NO: 36”, K25 is meant the lysine residue (K) at position 25 of SEQ ID NO: 36.

As used herein, the term “antibody” refers to any form of antibody that exhibits the desired biological activity (e.g., inhibiting binding of a ligand to its receptor or by inhibiting ligand-induced receptor signaling). “Antibody fragment” and “antigen-binding fragment” refer to antigen-binding fragments of an antibody and antibody analogs, which typically include at least a portion of the antigen-binding or variable regions (e.g. one or more CDRs) of the parent antibody. In some embodiments, the antibody is a monoclonal antibody. In some other embodiments, the antibody is a polyclonal antibody. The term “monoclonal antibody” as used herein refers to an antibody obtained from a population of substantially homogeneous antibodies—that is, the individual antibodies comprising the population are identical except for possible naturally occurring mutations that may be present in minor amounts. Monoclonal antibodies are highly specific and may be directed against a single antigenic site. Furthermore, in contrast to conventional (polyclonal) antibody preparations that typically include a number of different antibodies directed against a number of different determinants (epitopes), each monoclonal antibody is only directed against a single determinant on the antigen. The modifier “monoclonal” indicates the character of the antibody obtained from a substantially homogeneous population of antibodies, and is not to be construed as requiring any particular method to prepare the antibody. For example, the monoclonal antibodies for use in the present invention may be prepared by hybridoma or recombinant DNA methods. Monoclonal antibodies may include “chimeric” antibodies, humanized antibodies, or fully human antibodies. In some embodiments, the antibody forms part of a larger biomolecule, such as a fusion protein or an antibody-drug conjugate. An antibody fragment retains at least some of the binding specificity of the parent antibody. In general, an antibody fragment retains at least 10% of the parent binding activity when activity is expressed on a molar basis. Preferably, an antibody fragment retains at least 20%, 50%, 70%, 80%, 90%, 95%, or 100% or more of the binding affinity of the parent antibody for the target.

Thus, as used herein, examples of antibody fragments include, but are not limited to: Fab, Fab′, F(ab′)₂, and Fv fragments; diabodies; linear antibodies; single-chain antibody molecules, such as sc-Fv; nanobodies; domain antibodies; and multispecific antibodies (e.g., bispecific antibodies), CAR-T, BiTE, etc. formed from antibody fragments. “Fab fragment” is composed of one light chain and the CH1 and variable regions of one heavy chain. The heavy chain of a Fab molecule cannot form a disulfide bond with another heavy chain molecule. “Fab′ fragment” contains one light chain and a portion of one heavy chain that contains the VH domain and the CH1 domain and also the region between the CH1 and CH2 domains, such that an interchain disulfide bond can be formed between the two heavy chains of two Fab′ fragments to form a F(ab′)₂ molecule. “F(ab′)₂ fragment” contains two light chains and two heavy chains containing a portion of the constant region between the CH1 and CH2 domains, such that an interchain disulfide bond is formed between the two heavy chains. Thus, a F(ab′)₂ fragment is composed of two Fab′ fragments that are held together by a disulfide bond between the two heavy chains. “Fv region” comprises the variable regions from both the heavy and light chains but lacks the constant regions. “Single-chain Fv antibody” (or “scFv antibody”) refers to antibody fragments comprising the VH and VL domains of an antibody, wherein these domains are present in a single polypeptide chain. Generally, an Fv polypeptide comprises an additional polypeptide linker between the VH and VL domains, which enables the scFv to form the desired structure for antigen binding.

“Fc” or “Fc portion” or “Fc region” refers to a polypeptide that comprises the constant region of an antibody, excluding the first constant region immunoglobulin domain and, in some cases, part of the hinge. Thus, Fc refers to the last two constant region immunoglobulin domains of IgA, IgD, and IgG, the last three constant region immunoglobulin domains of IgE and IgM, and the flexible hinge N-termini of these domains. In some embodiments, amino acid modifications are made to the Fc region, for example, to alter binding to one or more FcγR receptors or FcRn receptors.

As used herein, the term “humanized antibody” refers to an antibody that comprises the CDRs of antibodies derived from mammals other than humans, and the framework regions (FR) and constant regions of a human antibody.

As used herein, a sequence “variant” refers to a sequence that differs from the indicated sequence at one or more amino acid residues but that retains the biological activity of the resulting molecule.

As used herein, the “% identity” between two sequences refers to a function of the number of identical positions shared by the sequences (i.e., % homology=number of identical positions/total number of positions×100), taking into account the number of gaps and the length of each of the gaps, which need to be introduced for optimal alignment of the two sequences. The comparison of sequences and the determination of % identity between two sequences may be accomplished using a mathematical algorithm.

“Variant having at least 85% sequence identity” refers to a sequence that is identical or substantially similar in biological activity and function to the indicated sequence (e.g., an amino acid sequence) but that has about 85%, about 90%, about 95%, about 96%, about 97%, about 98%, or about 99% sequence identity to the indicated sequence.

“Conservative substitution” refers to amino acid substitutions known to those skilled in the art, which are generally made without altering the biological activity of the resulting molecule. Generally, those skilled in the art recognize that single amino acid substitutions in non-essential regions of a polypeptide do not or do not substantially alter biological activity. “Do not or do not substantially alter” means that one or more aspects have no more than about 20%, about 15%, about 10%, about 9%, about 8%, about 7%, about 6%, about 5%, about 4%, about 3%, about 2%, or about 1% difference as compared to the compared subject when measured in the same manner or a similar manner.

“Variant with one to three conservative amino acid substitutions” means that, compared to the indicated sequence, the variant has one to three (e.g., one to two, two to three, one, two, or three, the same or different) amino acid substitutions known to those skilled in the art, which are generally made without altering or substantially altering the biological activity of the resulting molecule. When each CDR may be replaced by a variant with one to three conservative amino acid substitutions, each CDR is independently conservatively substituted by one to three amino acids. For example, LCDR2 may be replaced by a variant with one conservative amino acid substitution, HCDR2 may be replaced by a variant with two conservative amino acid substitutions, and HCDR1 and LCDR3 may not be substituted at all. When multiple CDRs are involved in conservative substitutions, all substitutions do not generally or do not substantially alter the biological activity of the antibody molecule. Generally, HCDR3 and LCDR3 are believed to play a more important role in antigen recognition than the other CDRs. Thus, in the case of substitutions, conservative substitutions are preferably made to the CDRs other than HCDR3 and LCDR3 in the present disclosure. In some embodiments, HCDR1, HCDR3, and LCDR3 are not substituted. Preferred amino acid substitutions include, but are not limited to: substitutions that (1) reduce susceptibility to proteolysis, (2) reduce susceptibility to oxidation, (3) alter binding affinity for forming protein complexes, and (4) provide or modify other physicochemical or functional properties of these analogs. Analogs can include various mutations of sequences other than naturally occurring peptide sequences. For example, single or multiple amino acid substitutions (preferably conservative amino acid substitutions) may be made in a naturally occurring sequence (preferably in a portion of the polypeptide outside the regions forming intermolecular contacts). A conservative amino acid substitution should not substantially alter the structural characteristics of the parent sequence (for example, an amino acid replacement should not tend to disrupt a helix that occurs in the parent sequence, or characterize other secondary structure types that disrupt the parent sequence).

It is contemplated that the binding domain of the monoclonal antibody or the antigen-binding fragment thereof of the present invention may carry a signal peptide, which is usually located at the N-terminus of the secreted protein and generally consists of 15-30 amino acids. After the signal peptide sequence is synthesized, it is recognized by the signal recognition particle (SRP), suspending or slowing down protein synthesis. The signal recognition particle carries the ribosome to the endoplasmic reticulum, where protein synthesis resumes. Under the guidance of the signal peptide, the newly synthesized protein enters the lumen of the endoplasmic reticulum, and the signal peptide sequence is cleaved off by the action of a signal peptidase. If a stop-transfer sequence exists at the C-terminus of the nascent peptide chain, it may not be cleaved off by the signal peptidase; for example, ovalbumin contains an internal signal peptide. Neither its precursor nor mature form undergoes cleavage by the signal peptidase.

“Specific” binding, when referring to ligand/receptor, antibody/antigen, or other binding pairs, refers to a binding reaction that determines the presence of the protein in a heterogeneous population of proteins and other biological agents. Thus, under the specified conditions, a particular ligand/antigen binds to a particular receptor/antibody and does not bind in significant amounts to other proteins present in the sample. “Specific binding” means that the monoclonal antibody or the antigen-binding fragment thereof of the present invention is capable of specifically interacting with at least two, three, four, five, six, seven, eight, or more amino acids of each human target molecule. “Specific binding” of an antibody is characterized primarily by two parameters: a qualitative parameter (the binding epitope or where the antibody binds) and a quantitative parameter (the binding affinity or binding strength). Antibody binding epitopes may be determined by FACS, peptide-spot epitope mapping, mass spectroscopy, or peptide ELISA. The strength of binding of an antibody to a particular epitope may be determined by Biacore and/or ELISA. Signal-to-noise ratios are often used as a representative method for measuring and calculating binding specificity. In such a signal-to-noise ratio, the signal represents the strength of binding of the antibody to the target epitope, whereas the noise represents the strength of binding of the antibody to other non-target epitopes. Preferably, a signal-to-noise ratio of about 50 for the target epitope may be taken as an indication that the antibody evaluated binds to the target epitope in a specific manner, i.e., “specific binding”. An antigen-binding protein (including antibodies) “specifically binds” to an antigen if the antigen-binding protein (including antibodies) binds to the antigen with a high binding affinity as determined by the affinity constant (KD) value. In some embodiments, the affinity constant KD is less than 10⁻⁹ M. The term “KD” as used herein refers to the affinity constant for a particular antibody-antigen interaction.

In the present invention, “about” means that a value is within an acceptable error range for the particular value determined by one of ordinary skill in the art; the value will depend in part on how it is measured or determined (i.e., the limitations of the measurement system). For example, “about” may mean within 1 or more than 1 standard deviation in each practice in the art. Alternatively, “about” or “substantially comprising” may mean a range of up to 20%. Furthermore, particularly for biological systems or processes, the terms may mean at most an order of magnitude or at most 5 times the value. Unless otherwise indicated, when a particular value appears in the present application and claims, the meaning of “about” or “substantially comprising” should be assumed to be within an acceptable error range for that particular value.

“Administration” and “treatment”, when referring to an animal, human, experimental subject, cell, tissue, organ, or biological fluid, refer to contacting an exogenous drug, therapeutic agent, diagnostic agent, or composition with the animal, human, subject, cell, tissue, organ, or biological fluid. “Administration” and “treatment” may refer, e.g., to therapeutic, pharmacokinetic, diagnostic, research, and experimental methods. Treating a cell encompasses contacting a reagent with the cell, as well as contacting a reagent with a fluid, wherein the fluid is in contact with the cell. “Administration” and “treatment” also mean in vitro and ex vivo treatment of a cell, e.g., by a reagent, a diagnostic agent, or a binding composition, or by another cell.

As used herein, the term “inhibit” or “treat” includes a postponement of development of the symptoms associated with a disease and/or a reduction in the severity of these symptoms that will or are expected to develop with the disease. The term also includes ameliorating existing symptoms, preventing additional symptoms, and ameliorating or preventing the underlying causes of these symptoms. Thus, the term indicates that a beneficial result has been conferred on a vertebrate subject with a disease.

The term “therapeutically effective amount” or “effective amount” as used herein refers to an amount of the monoclonal antibody binding to human CD38 or the antigen-binding fragment thereof of the present invention that, when administered alone or in combination with an additional therapeutic agent to a cell, tissue, or subject, is effective in preventing or ameliorating the disease or disorder to be treated. A therapeutically effective dose further refers to an amount of the compound that is sufficient to cause amelioration of symptoms, e.g., treatment, healing, prevention, or amelioration of a related medical state, or an increase in the rate of treatment, healing, prevention, or amelioration of the condition. When an individual is administered an active ingredient administered alone, a therapeutically effective amount refers to that ingredient alone. When a combination is administered, a therapeutically effective amount refers to a combined amount of the active ingredients that produce the therapeutic effect, whether administered in combination, sequentially, or simultaneously. A therapeutically effective amount will alleviate the symptoms usually by at least 10%; usually by at least 20%; preferably by at least about 30%; more preferably by at least 40%, and most preferably by at least 50%.

The terms “cancer”, “malignant disease”, “neoplasm”, “tumor”, and “carcinoma” are used interchangeably herein to mean cells that exhibit relatively abnormal, uncontrolled, and/or autonomous growth, so that they exhibit an abnormal growth phenotype characterized by a significant loss of control of cell proliferation. In general, cells of interest for detection or treatment in the present application include precancerous (e.g., benign), malignant, pre-metastatic, metastatic, and non-metastatic cells. The cancer of the present invention may be associated with any cancer expressing CD38.

As used herein, the term “pharmaceutically acceptable” applied to the carrier, diluent, or excipient used to formulate a composition as disclosed herein means that the carrier, diluent, or excipient must be compatible with the other ingredients of the composition and not deleterious to the recipient thereof.

### Anti-CD38 Monoclonal Antibody

In one aspect of the present invention, a monoclonal antibody binding to human CD38 or an antigen-binding fragment thereof of the present invention comprises three heavy chain complementarity-determining regions HCDR1, HCDR2, and HCDR3, and three light chain complementarity-determining regions LCDR1, LCDR2, and LCDR3, wherein: the amino acid sequence of the HCDR1 is set forth in SEQ ID NO: 1 or a variant thereof with one to three conservative amino acid substitutions; the amino acid sequence of the HCDR2 is set forth in SEQ ID NO: 2 or a variant thereof with one to three conservative amino acid substitutions; the amino acid sequence of the HCDR3 is set forth in SEQ ID NO: 6 or a variant thereof with one to three conservative amino acid substitutions; the amino acid sequence of the LCDR1 is set forth in SEQ ID NO: 7 or a variant thereof with one to three conservative amino acid substitutions; the amino acid sequence of the LCDR2 is set forth in SEQ ID NO: 9 or a variant thereof with one to three conservative amino acid substitutions; and the amino acid sequence of the LCDR3 is set forth in SEQ ID NO: 11 or a variant thereof with one to three conservative amino acid substitutions.

In one aspect of the present invention, a monoclonal antibody binding to human CD38 or an antigen-binding fragment thereof of the present invention comprises three heavy chain complementarity-determining regions HCDR1, HCDR2, and HCDR3, and three light chain complementarity-determining regions LCDR1, LCDR2, and LCDR3, wherein: the amino acid sequence of the HCDR1 is set forth in SEQ ID NO: 1 or a variant thereof with one to three conservative amino acid substitutions; the amino acid sequence of the HCDR2 is set forth in SEQ ID NO: 2 or a variant thereof with one to three conservative amino acid substitutions; the amino acid sequence of the HCDR3 is set forth in SEQ ID NO: 6; the amino acid sequence of the LCDR1 is set forth in SEQ ID NO: 7 or a variant thereof with one to three conservative amino acid substitutions; the amino acid sequence of the LCDR2 is set forth in SEQ ID NO: 9 or a variant thereof with one to three conservative amino acid substitutions; and the amino acid sequence of the LCDR3 is set forth in SEQ ID NO: 11.

In one aspect of the present invention, a monoclonal antibody binding to human CD38 or an antigen-binding fragment thereof of the present invention comprises three heavy chain complementarity-determining regions HCDR1, HCDR2, and HCDR3, and three light chain complementarity-determining regions LCDR1, LCDR2, and LCDR3, wherein: the amino acid sequence of the HCDR1 is set forth in SEQ ID NO: 1; the amino acid sequence of the HCDR2 is set forth in SEQ ID NO: 2 or a variant thereof with one to three conservative amino acid substitutions; the amino acid sequence of the HCDR3 is set forth in SEQ ID NO: 6; the amino acid sequence of the LCDR1 is set forth in SEQ ID NO: 7 or a variant thereof with one to three conservative amino acid substitutions; the amino acid sequence of the LCDR2 is set forth in SEQ ID NO: 9 or a variant thereof with one to three conservative amino acid substitutions; and the amino acid sequence of the LCDR3 is set forth in SEQ ID NO: 11.

In some embodiments, the monoclonal antibody binding to human CD38 or the antigen-binding fragment thereof comprises three heavy chain complementarity-determining regions HCDR1, HCDR2, and HCDR3, and three light chain complementarity-determining regions LCDR1, LCDR2, and LCDR3, wherein: the amino acid sequence of the HCDR1 is set forth in SEQ ID NO: 1; the amino acid sequence of the HCDR2 is set forth in SEQ ID NO: 2, SEQ ID NO: 3, SEQ ID NO: 4, or SEQ ID NO: 5; the amino acid sequence of the HCDR3 is set forth in SEQ ID NO: 6; the amino acid sequence of the LCDR1 is set forth in SEQ ID NO: 7 or SEQ ID NO: 8; the amino acid sequence of the LCDR2 is set forth in SEQ ID NO: 9 or SEQ ID NO: 10; and the amino acid sequence of the LCDR3 is set forth in SEQ ID NO: 11.

In some embodiments, the monoclonal antibody binding to human CD38 or the antigen-binding fragment thereof comprises the following complementarity-determining regions: a HCDR1, the amino acid sequence of which is set forth in SEQ ID NO: 1; a HCDR2, the amino acid sequence of which is set forth in SEQ ID NO: 2; a HCDR3, the amino acid sequence of which is set forth in SEQ ID NO: 6; a LCDR1, the amino acid sequence of which is set forth in SEQ ID NO: 7; a LCDR2, the amino acid sequence of which is set forth in SEQ ID NO: 9; and a LCDR3, the amino acid sequence of which is set forth in SEQ ID NO: 11.

In some embodiments, the monoclonal antibody binding to human CD38 or the antigen-binding fragment thereof comprises the following complementarity-determining regions: a HCDR1, the amino acid sequence of which is set forth in SEQ ID NO: 1; a HCDR2, the amino acid sequence of which is set forth in SEQ ID NO: 3; a HCDR3, the amino acid sequence of which is set forth in SEQ ID NO: 6; a LCDR1, the amino acid sequence of which is set forth in SEQ ID NO: 7; a LCDR2, the amino acid sequence of which is set forth in SEQ ID NO: 9; and a LCDR3, the amino acid sequence of which is set forth in SEQ ID NO: 11.

In some embodiments, the monoclonal antibody binding to human CD38 or the antigen-binding fragment thereof comprises the following complementarity-determining regions: a HCDR1, the amino acid sequence of which is set forth in SEQ ID NO: 1; a HCDR2, the amino acid sequence of which is set forth in SEQ ID NO: 2; a HCDR3, the amino acid sequence of which is set forth in SEQ ID NO: 6; a LCDR1, the amino acid sequence of which is set forth in SEQ ID NO: 8; a LCDR2, the amino acid sequence of which is set forth in SEQ ID NO: 10; and a LCDR3, the amino acid sequence of which is set forth in SEQ ID NO: 11.

In some embodiments, the monoclonal antibody binding to human CD38 or the antigen-binding fragment thereof comprises the following complementarity-determining regions: a HCDR1, the amino acid sequence of which is set forth in SEQ ID NO: 1; a HCDR2, the amino acid sequence of which is set forth in SEQ ID NO: 4; a HCDR3, the amino acid sequence of which is set forth in SEQ ID NO: 6; a LCDR1, the amino acid sequence of which is set forth in SEQ ID NO: 7; a LCDR2, the amino acid sequence of which is set forth in SEQ ID NO: 9; and a LCDR3, the amino acid sequence of which is set forth in SEQ ID NO: 11.

In some embodiments, the monoclonal antibody binding to human CD38 or the antigen-binding fragment thereof comprises the following complementarity-determining regions: a HCDR1, the amino acid sequence of which is set forth in SEQ ID NO: 1; a HCDR2, the amino acid sequence of which is set forth in SEQ ID NO: 5; a HCDR3, the amino acid sequence of which is set forth in SEQ ID NO: 6; a LCDR1, the amino acid sequence of which is set forth in SEQ ID NO: 7; a LCDR2, the amino acid sequence of which is set forth in SEQ ID NO: 9; and a LCDR3, the amino acid sequence of which is set forth in SEQ ID NO: 11.

In any of the above embodiments, any of the HCDR1, HCDR2, HCDR3, LCDR1, LCDR2, and LCDR3 is obtained according to the Kabat definition scheme.

In another aspect of the present invention, the present disclosure provides a monoclonal antibody binding to human CD38 or an antigen-binding fragment thereof, comprising a heavy chain variable region (VH) and a light chain variable region (VL), wherein the heavy chain variable region (VH) comprises a HCDR1, a HCDR2, and a HCDR3, and the light chain variable region (VL) comprises a LCDR1, a LCDR2, and a LCDR3; the HCDR1, HCDR2, HCDR3, LCDR1, LCDR2, and LCDR3 are identical to the HCDR1, HCDR2, HCDR3, LCDR1, LCDR2, and LCDR3, respectively, selected from any of the following groups:
(a) the HCDR1, HCDR2, and HCDR3 of a heavy chain variable region whose amino acid sequence is set forth in SEQ ID NO: 12, and the LCDR1, LCDR2, and LCDR3 of a light chain variable region whose amino acid sequence is set forth in any of SEQ ID NOs: 18, 19, 20, and 21;
(b) the HCDR1, HCDR2, and HCDR3 of a heavy chain variable region whose amino acid sequence is set forth in SEQ ID NO: 13, and the LCDR1, LCDR2, and LCDR3 of a light chain variable region whose amino acid sequence is set forth in SEQ ID NO: 18 or 19;
(c) the HCDR1, HCDR2, and HCDR3 of a heavy chain variable region whose amino acid sequence is set forth in SEQ ID NO: 14, and the LCDR1, LCDR2, and LCDR3 of a light chain variable region whose amino acid sequence is set forth in any of SEQ ID NOs: 18, 20, and 21;
(d) the HCDR1, HCDR2, and HCDR3 of a heavy chain variable region whose amino acid sequence is set forth in SEQ ID NO: 15, and the LCDR1, LCDR2, and LCDR3 of a light chain variable region whose amino acid sequence is set forth in SEQ ID NO: 18;
(e) the HCDR1, HCDR2, and HCDR3 of a heavy chain variable region whose amino acid sequence is set forth in SEQ ID NO: 16, and the LCDR1, LCDR2, and LCDR3 of a light chain variable region whose amino acid sequence is set forth in any of SEQ ID NOs: 18, 19, 20, and 21;
(f) the HCDR1, HCDR2, and HCDR3 of a heavy chain variable region whose amino acid sequence is set forth in SEQ ID NO: 17, and the LCDR1, LCDR2, and LCDR3 of a light chain variable region whose amino acid sequence is set forth in SEQ ID NO: 18 or 19; and
(g) the HCDR1, HCDR2, and HCDR3 of a heavy chain variable region whose amino acid sequence is set forth in SEQ ID NO: 23, and the LCDR1, LCDR2, and LCDR3 of a light chain variable region whose amino acid sequence is set forth in SEQ ID NO: 24.

In another aspect of the present invention described above, each group of CDRs is defined according to any of the Kabat, Chothia, IMGT, Contact, and AbM definition schemes. In a preferred embodiment of that aspect, each group of CDRs is defined according to any of the Chothia, IMGT, Contact, and AbM definition schemes. In a preferred embodiment of that aspect, each group of CDRs is defined according to the Kabat definition scheme. In a preferred embodiment of that aspect, each group of CDRs is defined according to the Chothia definition scheme. In a preferred embodiment of that aspect, each group of CDRs is defined according to the IMGT definition scheme. In a preferred embodiment of that aspect, each group of CDRs is defined according to the Contact definition scheme. In a preferred embodiment of that aspect, each group of CDRs is defined according to the AbM definition scheme.

In another aspect of the present invention, the monoclonal antibody binding to human CD38 or the antigen-binding fragment thereof of the present invention comprises: (1) a VH, comprising an amino acid sequence selected from SEQ ID NO: 12, SEQ ID NO: 13, SEQ ID NO: 14, SEQ ID NO: 15, SEQ ID NO: 16, or SEQ ID NO: 17 or a variant having at least 85% sequence identity thereto; and (2) a VL, comprising an amino acid sequence selected from SEQ ID NO: 18, SEQ ID NO: 19, SEQ ID NO: 20, or SEQ ID NO: 21 or a variant having at least 85% sequence identity thereto.

In some embodiments, the monoclonal antibody binding to human CD38 or the antigen-binding fragment thereof comprises the following variable regions: a VH, comprising the amino acid sequence set forth in SEQ ID NO: 12 or a variant having at least 85% sequence identity thereto; and a VL, comprising the amino acid sequence set forth in SEQ ID NO: 18 or a variant having at least 85% sequence identity thereto.

In some embodiments, the monoclonal antibody binding to human CD38 or the antigen-binding fragment thereof comprises the following variable regions: a VH, comprising the amino acid sequence set forth in SEQ ID NO: 13 or a variant having at least 85% sequence identity thereto; and a VL, comprising the amino acid sequence set forth in SEQ ID NO: 18 or a variant having at least 85% sequence identity thereto.

In some embodiments, the monoclonal antibody binding to human CD38 or the antigen-binding fragment thereof comprises the following variable regions: a VH, comprising the amino acid sequence set forth in SEQ ID NO: 12 or a variant having at least 85% sequence identity thereto; and a VL, comprising the amino acid sequence set forth in SEQ ID NO: 19 or a variant having at least 85% sequence identity thereto.

In some embodiments, the monoclonal antibody binding to human CD38 or the antigen-binding fragment thereof comprises the following variable regions: a VH, comprising the amino acid sequence set forth in SEQ ID NO: 13 or a variant having at least 85% sequence identity thereto; and a VL, comprising the amino acid sequence set forth in SEQ ID NO: 19 or a variant having at least 85% sequence identity thereto.

In some embodiments, the monoclonal antibody binding to human CD38 or the antigen-binding fragment thereof comprises the following variable regions: a VH, comprising the amino acid sequence set forth in SEQ ID NO: 12 or a variant having at least 85% sequence identity thereto; and a VL, comprising the amino acid sequence set forth in SEQ ID NO: 20 or a variant having at least 85% sequence identity thereto.

In some embodiments, the monoclonal antibody binding to human CD38 or the antigen-binding fragment thereof comprises the following variable regions: a VH, comprising the amino acid sequence set forth in SEQ ID NO: 12 or a variant having at least 85% sequence identity thereto; and a VL, comprising the amino acid sequence set forth in SEQ ID NO: 21 or a variant having at least 85% sequence identity thereto.

In some embodiments, the monoclonal antibody binding to human CD38 or the antigen-binding fragment thereof comprises the following variable regions: a VH, comprising the amino acid sequence set forth in SEQ ID NO: 14 or a variant having at least 85% sequence identity thereto; and a VL, comprising the amino acid sequence set forth in SEQ ID NO: 18 or a variant having at least 85% sequence identity thereto.

In some embodiments, the monoclonal antibody binding to human CD38 or the antigen-binding fragment thereof comprises the following variable regions: a VH, comprising the amino acid sequence set forth in SEQ ID NO: 15 or a variant having at least 85% sequence identity thereto; and a VL, comprising the amino acid sequence set forth in SEQ ID NO: 18 or a variant having at least 85% sequence identity thereto.

In some embodiments, the monoclonal antibody binding to human CD38 or the antigen-binding fragment thereof comprises the following variable regions: a VH, comprising the amino acid sequence set forth in SEQ ID NO: 14 or a variant having at least 85% sequence identity thereto; and a VL, comprising the amino acid sequence set forth in SEQ ID NO: 19 or a variant having at least 85% sequence identity thereto.

In some embodiments, the monoclonal antibody binding to human CD38 or the antigen-binding fragment thereof comprises the following variable regions: a VH, comprising the amino acid sequence set forth in SEQ ID NO: 15 or a variant having at least 85% sequence identity thereto; and a VL, comprising the amino acid sequence set forth in SEQ ID NO: 19 or a variant having at least 85% sequence identity thereto.

In some embodiments, the monoclonal antibody binding to human CD38 or the antigen-binding fragment thereof comprises the following variable regions: a VH, comprising the amino acid sequence set forth in SEQ ID NO: 14 or a variant having at least 85% sequence identity thereto; and a VL, comprising the amino acid sequence set forth in SEQ ID NO: 20 or a variant having at least 85% sequence identity thereto.

In some embodiments, the monoclonal antibody binding to human CD38 or the antigen-binding fragment thereof comprises the following variable regions: a VH, comprising the amino acid sequence set forth in SEQ ID NO: 14 or a variant having at least 85% sequence identity thereto; and a VL, comprising the amino acid sequence set forth in SEQ ID NO: 21 or a variant having at least 85% sequence identity thereto.

In some embodiments, the monoclonal antibody binding to human CD38 or the antigen-binding fragment thereof comprises the following variable regions: a VH, comprising the amino acid sequence set forth in SEQ ID NO: 16 or a variant having at least 85% sequence identity thereto; and a VL, comprising the amino acid sequence set forth in SEQ ID NO: 18 or a variant having at least 85% sequence identity thereto.

In some embodiments, the monoclonal antibody binding to human CD38 or the antigen-binding fragment thereof comprises the following variable regions: a VH, comprising the amino acid sequence set forth in SEQ ID NO: 17 or a variant having at least 85% sequence identity thereto; and a VL, comprising the amino acid sequence set forth in SEQ ID NO: 18 or a variant having at least 85% sequence identity thereto.

In some embodiments, the monoclonal antibody binding to human CD38 or the antigen-binding fragment thereof comprises the following variable regions: a VH, comprising the amino acid sequence set forth in SEQ ID NO: 16 or a variant having at least 85% sequence identity thereto; and a VL, comprising the amino acid sequence set forth in SEQ ID NO: 19 or a variant having at least 85% sequence identity thereto.

In some embodiments, the monoclonal antibody binding to human CD38 or the antigen-binding fragment thereof comprises the following variable regions: a VH, comprising the amino acid sequence set forth in SEQ ID NO: 17 or a variant having at least 85% sequence identity thereto; and a VL, comprising the amino acid sequence set forth in SEQ ID NO: 19 or a variant having at least 85% sequence identity thereto.

In some embodiments, the monoclonal antibody binding to human CD38 or the antigen-binding fragment thereof comprises the following variable regions: a VH, comprising the amino acid sequence set forth in SEQ ID NO: 16 or a variant having at least 85% sequence identity thereto; and a VL, comprising the amino acid sequence set forth in SEQ ID NO: 20 or a variant having at least 85% sequence identity thereto.

In some embodiments, the monoclonal antibody binding to human CD38 or the antigen-binding fragment thereof comprises the following variable regions: a VH, comprising the amino acid sequence set forth in SEQ ID NO: 16 or a variant having at least 85% sequence identity thereto; and a VL, comprising the amino acid sequence set forth in SEQ ID NO: 21 or a variant having at least 85% sequence identity thereto.

In some embodiments, the monoclonal antibody binding to human CD38 or the antigen-binding fragment thereof comprises the following variable regions: a VH, comprising the amino acid sequence set forth in SEQ ID NO: 23 or a variant having at least 85% sequence identity thereto; and a VL, comprising the amino acid sequence set forth in SEQ ID NO: 24 or a variant having at least 85% sequence identity thereto.

In some embodiments, the monoclonal antibody binding to human CD38 or the antigen-binding fragment thereof comprises a heavy chain constant region and a light chain constant region, wherein the heavy chain constant region comprises the amino acid sequence set forth in SEQ ID NO: 25, and the light chain constant region comprises the amino acid sequence set forth in SEQ ID NO: 33.

In some embodiments, an Fc portion of any of the above monoclonal antibodies is modified to enhance binding to FcγRIIIa (V) and/or FcγRIIIa (F). In some embodiments, the Fc portion of the monoclonal antibody comprises the amino acid sequence set forth in SEQ ID NO: 27 or a variant having at least 85% sequence identity thereto.

In another aspect of the present invention, the monoclonal antibody binding to human CD38 or the antigen-binding fragment thereof of the present invention comprises: (1) a heavy chain, comprising the amino acid sequence set forth in SEQ ID NO: 28, SEQ ID NO: 29, SEQ ID NO: 30, or SEQ ID NO: 31 or a variant having at least 85% sequence identity thereto; and (2) a light chain, comprising the amino acid sequence set forth in SEQ ID NO: 34 or SEQ ID NO: 35 or a variant having at least 85% sequence identity thereto.

In some embodiments, the monoclonal antibody binding to human CD38 or the antigen-binding fragment thereof comprises: a heavy chain, comprising the amino acid sequence set forth in SEQ ID NO: 28 or a variant having at least 85% sequence identity thereto; and a light chain, comprising the amino acid sequence set forth in SEQ ID NO: 34 or a variant having at least 85% sequence identity thereto.

In some embodiments, the monoclonal antibody binding to human CD38 or the antigen-binding fragment thereof comprises: a heavy chain, comprising the amino acid sequence set forth in SEQ ID NO: 28 or a variant having at least 85% sequence identity thereto; and a light chain, comprising the amino acid sequence set forth in SEQ ID NO: 35 or a variant having at least 85% sequence identity thereto.

In some embodiments, the monoclonal antibody binding to human CD38 or the antigen-binding fragment thereof comprises: a heavy chain, comprising the amino acid sequence set forth in SEQ ID NO: 29 or a variant having at least 85% sequence identity thereto; and a light chain, comprising the amino acid sequence set forth in SEQ ID NO: 34 or a variant having at least 85% sequence identity thereto.

In some embodiments, the monoclonal antibody binding to human CD38 or the antigen-binding fragment thereof comprises: a heavy chain, comprising the amino acid sequence set forth in SEQ ID NO: 30 or a variant having at least 85% sequence identity thereto; and a light chain, comprising the amino acid sequence set forth in SEQ ID NO: 34 or a variant having at least 85% sequence identity thereto.

In some embodiments, the monoclonal antibody binding to human CD38 or the antigen-binding fragment thereof comprises: a heavy chain, comprising the amino acid sequence set forth in SEQ ID NO: 30 or a variant having at least 85% sequence identity thereto; and a light chain, comprising the amino acid sequence set forth in SEQ ID NO: 35 or a variant having at least 85% sequence identity thereto.

In some embodiments, the monoclonal antibody binding to human CD38 or the antigen-binding fragment thereof comprises: a heavy chain, comprising the amino acid sequence set forth in SEQ ID NO: 31 or a variant having at least 85% sequence identity thereto; and a light chain, comprising the amino acid sequence set forth in SEQ ID NO: 34 or a variant having at least 85% sequence identity thereto.

In some embodiments, the monoclonal antibody is of an IgG type. In some embodiments, the monoclonal antibody is of the IgG1 type.

In some embodiments, the monoclonal antibody or the antigen-binding fragment thereof does not bind to healthy human erythrocytes. In some embodiments, the monoclonal antibody or the antigen-binding fragment thereof is capable of binding to healthy human PBMCs. In some embodiments, the monoclonal antibody or the antigen-binding fragment thereof does not bind to healthy human erythrocytes and is capable of binding to healthy human PBMCs. In some embodiments, the present disclosure provides a monoclonal antibody or an antigen-binding fragment thereof, which binds to SEQ ID NO: 22 but does not bind to healthy human erythrocytes. In some embodiments, the present disclosure provides a monoclonal antibody or an antigen-binding fragment thereof, which binds to SEQ ID NO: 22 but does not bind to healthy human erythrocytes, and competes for binding to human CD38 with any of the monoclonal antibodies or antigen-binding fragments provided by the present disclosure. In some embodiments, the present disclosure provides a monoclonal antibody or an antigen-binding fragment thereof, which binds to SEQ ID NO: 22 but does not bind to healthy human erythrocytes, and competes with FTL004-1, FTL004-6, or FTL004-7 for binding to human CD38. In some embodiments, the present disclosure provides a monoclonal antibody or an antigen-binding fragment thereof, which binds to SEQ ID NO: 22 but does not bind to healthy human erythrocytes, and competes with FTL004-1 for binding to human CD38.

In some embodiments, the monoclonal antibody or the antigen-binding fragment thereof has a 10⁻⁹ M level affinity constant Kd as determined by Biacore. In some embodiments, the monoclonal antibody or the antigen-binding fragment thereof has an affinity constant Kd of 1.42×10⁻⁹ M to 2.78×10⁻⁹ M as determined by Biacore.

In some embodiments, the monoclonal antibody or the antigen-binding fragment thereof binds to SEQ ID NO: 22. In some embodiments, the monoclonal antibody or the antigen-binding fragment thereof binds to a different antigenic epitope than isatuximab does. In some embodiments, the monoclonal antibody or the antigen-binding fragment thereof binds to a different antigenic epitope than daratumumab does. In some embodiments, the monoclonal antibody or the antigen-binding fragment thereof binds to a different antigenic epitope than isatuximab and daratumumab do. In some embodiments, the present disclosure provides a monoclonal antibody or an antigen-binding fragment thereof, which binds to SEQ ID NO: 22 and binds to a different antigenic epitope than isatuximab and daratumumab do. In some embodiments, the present disclosure provides a monoclonal antibody or an antigen-binding fragment thereof, which binds to SEQ ID NO: 22, binds to a different antigenic epitope than isatuximab and daratumumab do, and does not bind to healthy human erythrocytes.

In some embodiments, the present disclosure provides a monoclonal antibody or an antigen-binding fragment thereof, which competes for binding to human CD38 with any of the monoclonal antibodies or antigen-binding fragments provided by the present disclosure. In some embodiments, the monoclonal antibody or the antigen-binding fragment thereof provided by the present disclosure competes for binding to human CD38 with an antibody selected from any of the following: H2, FTL004-hu-1, FTL004-hu-2, FTL004-hu-3, FTL004-hu-4, FTL004-hu-5, FTL004-hu-6, FTL004-hu-7, FTL004-hu-8, FTL004-hu-9, FTL004-hu-10, FTL004-hu-11, FTL004-hu-12, FTL004-hu-13, FTL004-hu-14, FTL004-hu-15, FTL004-hu-16, FTL004-hu-17, FTL004-hu-18, FTL004-1, FTL004-6, and FTL004-7. In some embodiments, the monoclonal antibody or the antigen-binding fragment thereof provided by the present disclosure competes with FTL004-1, FTL004-6, or FTL004-7 for binding to human CD38. In some embodiments, the monoclonal antibody or the antigen-binding fragment thereof provided by the present disclosure competes with FTL004-1 for binding to human CD38.

In some embodiments, the present disclosure provides a monoclonal antibody or an antigen-binding fragment thereof, which binds to SEQ ID NO: 22, binds to a different antigenic epitope than isatuximab and daratumumab do, but does not bind to healthy human erythrocytes, and competes for binding to human CD38 with any of the monoclonal antibodies or antigen-binding fragments provided by the present disclosure.

In some embodiments, the present disclosure provides a monoclonal antibody or an antigen-binding fragment thereof, which binds to SEQ ID NO: 22, binds to a different antigenic epitope than isatuximab and daratumumab do, but does not bind to healthy human erythrocytes, and competes with FTL004-1, FTL004-6, or FTL004-7 for binding to human CD38.

In some embodiments, the present disclosure provides a monoclonal antibody or an antigen-binding fragment thereof, which binds to SEQ ID NO: 22, binds to a different antigenic epitope than isatuximab and daratumumab do, but does not bind to healthy human erythrocytes, and competes with FTL004-1 for binding to human CD38.

Another aspect of the present disclosure provides a monoclonal antibody specifically binding to human CD38, or an antigen binding fragment thereof, wherein the monoclonal antibody binds to an extracellular domain of CD38 at amino acid residues comprising at least one amino acid residue selected from a group consisting of K25, E28, 129, M33, and R34, wherein the amino acid residue is numbered according to the amino acid sequence of the extracellular domain of CD38 as shown in SEQ ID NO: 36. In some embodiments, the monoclonal antibody or an antigen binding fragment thereof binds to an extracellular domain of CD38 at amino acid residues comprising K25, E28, 129, M33 and R34. In some embodiments, the monoclonal antibody or an antigen binding fragment thereof further binds to an extracellular domain of CD38 at amino acid residues comprising at least one of P31, E32, H35, K67, T70, Q71, T72 and V73. In some embodiments, the monoclonal antibody or an antigen binding fragment thereof further binds to an extracellular domain of CD38 at amino acid residues comprising P31, E32, H35, K67, T70, Q71, T72 and V73.

In some embodiments, the monoclonal antibody or an antigen binding fragment thereof binds to an extracellular domain of CD38 at amino acid residues consisting of K25, E28, 129, P31, E32, M33, R34, H3, K67, T70, Q71, T72 and V73. In some embodiments, the antigen binding fragment is selected from the group consisting of Fv, scFv, (scFv) 2, Fab, Fab′ and F(ab′)₂. In some embodiments, the monoclonal antibody or an antigen binding fragment thereof does not bind to healthy human erythrocytes.

When using recombinant techniques, the antibody can be produced intracellularly or in the periplasmic space, or directly secreted into the medium. If the antibody is produced intracellularly, as a first step, particulate debris (host cells or lysed fragments) is removed, for example, by centrifugation or ultrafiltration. Where the antibody is secreted into the medium, the supernatant from the expression system is generally concentrated first using a commercially available protein concentration filter (e.g., an Amicon or Millipore Pellicon ultrafiltration unit). A protease inhibitor (e.g., PMSF) may be used in any of the aforementioned steps to inhibit proteolysis, and an antibiotic may be used to prevent the growth of adventitious contaminants.

Depending on the antibody to be recovered, other techniques for protein purification, such as fractionation on an ion-exchange column, ethanol precipitation, reversed-phase HPLC, silica gel chromatography, anion or cation exchange resin (e.g., polyaspartic acid column) chromatography, chromatofocusing, SDS-PAGE, and ammonium sulfate precipitation, may also be utilized. In one embodiment, the glycoprotein may be purified by letting the glycoprotein be adsorbed onto a lectin substrate (e.g. a lectin affinity column) to remove fucose-containing glycoproteins from the preparation and thereby enrich for fucose-free glycoproteins.

To express the heavy and/or light chains of the monoclonal antibody or the antigen-binding fragment thereof of the present invention, polynucleotides encoding the heavy and/or light chains are inserted into an expression vector such that the genes are operably ligated to transcriptional and translational sequences. Expression vectors are all vectors that those skilled in the art will know how to use to conveniently ensure the expression of the heavy and/or light chains. In some embodiments, the vector is a viral vector or non-viral vector. In some embodiments, the non-viral vector is selected from: a plasmid, a liposome, a reverse transcription element, a transposon, and an exosome. In some embodiments, the viral vector is selected from: a retrovirus, an adenovirus, an adeno-associated virus, a herpes simplex virus, a vaccinia virus, a baculovirus, and a lentivirus. One skilled in the art will realize that the polynucleotides encoding the heavy and light chains can be cloned into different vectors, or in the same vector. In a preferred embodiment, the polynucleotides are cloned in the same vector.

Thus, the present disclosure also provides a nucleotide sequence encoding the monoclonal antibody or the antigen-binding fragment thereof of the present invention. In one embodiment, the nucleotide molecule encodes the heavy and/or light chains of the monoclonal antibody or the antigen-binding fragment thereof of the present invention. In a preferred embodiment, a single nucleic acid encodes the heavy chain of the monoclonal antibody or the antigen-binding fragment thereof of the present invention, and another nucleic acid molecule encodes the light chain of the monoclonal antibody or the antigen-binding fragment thereof of the present invention.

Another aspect of the present invention provides a polynucleotide, encoding a polypeptide having an amino acid sequence selected from SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, and 35. In a preferred embodiment, the polypeptide encoded by the polynucleotide of the present invention is selected from SEQ ID NOs: 1, 2, 6, 7, 8, 9, 10, 11, 12, 14, 18, 21, 23, 24, 25, 26, 27, 28, 29, 30, 31, 33, 34, and 35. The present invention is not limited to the polynucleotide itself but also includes all polynucleotides that exhibit at least 80% identity to the polynucleotide.

The present invention provides a vector comprising the nucleotide sequence of the present invention. In one embodiment, the vector comprises a nucleotide encoding the monoclonal antibody or the antigen-binding fragment thereof of the present invention. In another embodiment, the nucleotide encodes the heavy chain of the monoclonal antibody or the antigen-binding fragment thereof of the present invention. In another embodiment, the nucleotide encodes the light chain of the monoclonal antibody or the antigen-binding fragment thereof of the present invention. The present invention also provides a vector comprising polynucleotide molecules encoding a fusion protein, a modified antibody, an antibody fragment, and a probe thereof.

The polynucleotides of the present invention and vectors comprising these molecules can be used to transform an appropriate mammalian host cell. Transformation can be performed by any known method for introducing a polynucleotide into a cell host, which is well known to those skilled in the art.

### Pharmaceutical Composition

Another aspect of the present disclosure provides a pharmaceutical composition, comprising any of the monoclonal antibodies or antigen-binding fragments thereof described above in the present disclosure, and a pharmaceutically acceptable carrier.

“Pharmaceutical composition” refers to a pharmaceutical formulation for use in humans. The pharmaceutical composition comprises a suitable formulation of the monoclonal antibody binding to human CD38 or the antigen-binding fragment thereof of the present invention, and a carrier, a stabilizer, and/or an excipient. The present invention provides a pharmaceutical formulation comprising the monoclonal antibody or the antigen-binding fragment thereof of the present invention. In some embodiments, the pharmaceutical composition of the present invention comprises the monoclonal antibody or the antigen-binding fragment thereof of the present invention, and a pharmaceutically acceptable carrier. To prepare a pharmaceutical composition or sterile composition, the antibody or the antigen-binding fragment thereof is mixed with a pharmaceutically acceptable carrier or excipient. Formulations of therapeutic and diagnostic drugs in the form of, e.g., lyophilized powders, slurries, aqueous solutions, or suspensions may be prepared by mixing with physiologically acceptable carriers, excipients, or stabilizers.

The toxicity and therapeutic efficacy of an antibody composition, administered alone or in combination with an immunosuppressive agent, can be determined in cell cultures or experimental animals by a standard pharmaceutical method, e.g., a method for determining the LD50 (the dose lethal to 50% of the population) and the ED50 (the dose therapeutically effective in 50% of the population). The dose ratio between toxic and therapeutic effects is a therapeutic index, and it can be expressed as the ratio of LD50 to ED50. The data obtained from these cell culture assays and animal studies can be used in formulating a dosage range for use in humans. The dosage of the compound falls preferably within a range of circulating concentrations that include the ED50 with little or no toxicity. The dosage may vary within this range depending on the dosage form employed and the route of administration utilized.

Suitable routes of administration include parenteral administration (e.g., intramuscular, intravenous, or subcutaneous administration) and oral administration. An antibody used in a pharmaceutical composition or to practice the method of the present invention may be administered in a variety of conventional ways, such as oral ingestion, inhalation, topical application, or cutaneous, subcutaneous, intraperitoneal, parenteral, intraarterial, or intravenous injection. In one embodiment, the binding compound of the present invention is administered intravenously. In another embodiment, the binding compound of the present invention is administered subcutaneously. Alternately, one may administer the antibody in a local rather than systemic manner (often in a depot or sustained-release formulation), for example, via injection of the antibody directly into the site of action. Furthermore, one may administer the antibody in a targeted drug delivery system.

The appropriate dose is determined by a clinician, e.g., using parameters or factors known in the art or suspected to affect treatment or expected to affect treatment. Generally, the dose begins with an amount somewhat less than the optimal dose and is thereafter increased by small increments until the desired or optimal effect is achieved relative to any adverse side effects. Important diagnostic measures include those of, e.g., inflammatory symptoms or the level of inflammatory cytokines produced.

Antibodies, antibody fragments, and cytokines may be provided by continuous infusion, or by administrations at intervals of, e.g., one day, one week, or 1-7 times every week. Doses may be provided intravenously, subcutaneously, intraperitoneally, transdermally, topically, orally, nasally, rectally, intramuscularly, intracerebrally, intraspinally, or by inhalation. A preferred dosage regimen is one that includes the maximal dose or frequency of administration that avoids significant undesirable side effects. A total weekly dose is generally at least 0.05 μg/kg body weight, more generally at least 0.2 μg/kg, most generally at least 0.5 μg/kg, typically at least 1 μg/kg, more typically at least 10 μg/kg, most typically at least 109 μg/kg, preferably at least 0.2 mg/kg, more preferably at least 1.0 mg/kg, most preferably at least 2.0 mg/kg, ideally at least 10 mg/kg, more ideally at least 25 mg/kg, and most ideally at least 50 mg/kg. The desired dose of a small-molecule therapeutic agent, e.g., a peptide mimetic, natural product, or organic chemical agent, is about the same as the dose of an antibody or polypeptide, on a mole/kg basis.

The pharmaceutical composition of the present invention may also comprise other agents, including but not limited to a cytotoxic agent, a cytostatic agent, an anti-angiogenic or anti-metabolic drug, a tumor-targeting drug, an immune stimulating or immune modulating agent, or an antibody conjugated to a cytotoxic agent, a cytostatic agent, or other toxic drugs. The pharmaceutical composition may also be administered with other therapeutic modalities (e.g., surgery, chemotherapy, and radiation). Typical veterinary, experimental, or research subjects include monkeys, dogs, cats, rats, mice, rabbits, guinea pigs, horses, and humans.

Specifically, the monoclonal antibody binding to human CD38 or the antigen-binding fragment thereof of the present invention may be used in combination with a second therapeutic agent for treating the same cancer. In a specific embodiment, the second therapeutic agent and the monoclonal antibody or the antigen-binding fragment thereof of the present invention are administered at substantially the same time. An individual will sometimes use the second therapeutic agent concurrently with the monoclonal antibody or the antigen-binding fragment thereof of the present invention. In one embodiment, the second therapeutic agent or other agents typically administered to a cancer patient and the monoclonal antibody or the antigen-binding fragment thereof of the present invention may be combined into a pharmaceutical composition; in other specific embodiments, the two are administered separately.

The second therapeutic agent is any agent that is advantageously combined with an anti-CD38 antibody. Exemplary agents that may be advantageously combined with an anti-CD38 antibody include, but are not limited to, other agents that inhibit CD38 activity (including other antibodies or antigen-binding fragments thereof, peptide inhibitors, small-molecule antagonists, etc.) and/or agents that interfere with signaling upstream or downstream of CD38. The second therapeutic agent may be a chemotherapeutic, radiotherapeutic, or biological agent. The biological agent may be a monoclonal antibody, an ADC, an oncolytic virus, or CAR-T.

### Treatment Method and Use

Another aspect of the present disclosure provides use of the monoclonal antibody or the antigen-binding fragment thereof or the pharmaceutical composition of the present invention in the preparation of a medicament for treating a CD38-positive cancer or an autoimmune disease.

In some embodiments, the monoclonal antibody comprises three heavy chain complementarity-determining regions HCDR1, HCDR2, and HCDR3, and three light chain complementarity-determining regions LCDR1, LCDR2, and LCDR3, wherein: the amino acid sequence of the HCDR1 is set forth in SEQ ID NO: 1 or a variant thereof with one to three conservative amino acid substitutions; the amino acid sequence of the HCDR2 is set forth in SEQ ID NO: 2 or a variant thereof with one to three conservative amino acid substitutions; the amino acid sequence of the HCDR3 is set forth in SEQ ID NO: 6 or a variant thereof with one to three conservative amino acid substitutions; the amino acid sequence of the LCDR1 is set forth in SEQ ID NO: 7 or a variant thereof with one to three conservative amino acid substitutions; the amino acid sequence of the LCDR2 is set forth in SEQ ID NO: 9 or a variant thereof with one to three conservative amino acid substitutions; and the amino acid sequence of the LCDR3 is set forth in SEQ ID NO: 11 or a variant thereof with one to three conservative amino acid substitutions. In some embodiments, the amino acid sequence of the HCDR1 is set forth in SEQ ID NO: 1; the amino acid sequence of the HCDR2 is set forth in SEQ ID NO: 2, SEQ ID NO: 3, SEQ ID NO: 4, or SEQ ID NO: 5; the amino acid sequence of the HCDR3 is set forth in SEQ ID NO: 6; the amino acid sequence of the LCDR1 is set forth in SEQ ID NO: 7 or SEQ ID NO: 8; the amino acid sequence of the LCDR2 is set forth in SEQ ID NO: 9 or SEQ ID NO: 10; and the amino acid sequence of the LCDR3 is set forth in SEQ ID NO: 11.

In some embodiments, the monoclonal antibody comprises a heavy chain variable region (VH) and a light chain variable region (VL), wherein:
(a) the VH comprises the amino acid sequence set forth in SEQ ID NO: 12 or a variant having at least 85% sequence identity thereto; the VL comprises the amino acid sequence set forth in SEQ ID NO: 18 or a variant having at least 85% sequence identity thereto;
(b) the VH comprises the amino acid sequence set forth in SEQ ID NO: 12 or a variant having at least 85% sequence identity thereto; the VL comprises the amino acid sequence set forth in SEQ ID NO: 21 or a variant having at least 85% sequence identity thereto; or
(c) the VH comprises the amino acid sequence set forth in SEQ ID NO: 14 or a variant having at least 85% sequence identity thereto; the VL comprises the amino acid sequence set forth in SEQ ID NO: 18 or a variant having at least 85% sequence identity thereto.

In some embodiments, the monoclonal antibody comprises a heavy chain and a light chain, wherein:
(a) the heavy chain comprises the amino acid sequence set forth in SEQ ID NO: 28 or a variant having at least 85% sequence identity thereto; the light chain comprises the amino acid sequence set forth in SEQ ID NO: 34 or a variant having at least 85% sequence identity thereto;
(b) the heavy chain comprises the amino acid sequence set forth in SEQ ID NO: 28 or a variant having at least 85% sequence identity thereto; the light chain comprises the amino acid sequence set forth in SEQ ID NO: 35 or a variant having at least 85% sequence identity thereto;
(c) the heavy chain comprises the amino acid sequence set forth in SEQ ID NO: 29 or a variant having at least 85% sequence identity thereto; the light chain comprises the amino acid sequence set forth in SEQ ID NO: 34 or a variant having at least 85% sequence identity thereto;
(d) the heavy chain comprises the amino acid sequence set forth in SEQ ID NO: 30 or a variant having at least 85% sequence identity thereto; the light chain comprises the amino acid sequence set forth in SEQ ID NO: 34 or a variant having at least 85% sequence identity thereto;
(e) the heavy chain comprises the amino acid sequence set forth in SEQ ID NO: 30 or a variant having at least 85% sequence identity thereto; the light chain comprises the amino acid sequence set forth in SEQ ID NO: 35 or a variant having at least 85% sequence identity thereto; or
(f) the heavy chain comprises the amino acid sequence set forth in SEQ ID NO: 31 or a variant having at least 85% sequence identity thereto; the light chain comprises the amino acid sequence set forth in SEQ ID NO: 34 or a variant having at least 85% sequence identity thereto.

In some embodiments, the monoclonal antibody binds to CD38, wherein the monoclonal antibody binds to an extracellular domain of CD38 at amino acid residues comprising at least one amino acid residue selected from a group consisting of K25, E28, 129, M33, and R34, wherein the amino acid residue is numbered according to the amino acid sequence of the extracellular domain of CD38 as shown in SEQ ID NO: 36. In some embodiments, the monoclonal antibody binds to CD38, wherein the monoclonal antibody binds to an extracellular domain of CD38 at amino acid residues consisting of K25, E28, 129, P31, E32, M33, R34, H3, K67, T70, Q71, T72 and V73.

Another aspect of the present disclosure provides a method for treating a CD38-positive cancer or an autoimmune disease with the monoclonal antibody or the antigen-binding fragment thereof or the pharmaceutical composition of the present invention, the method comprising administering to a subject a therapeutically effective amount of any of the monoclonal antibodies or antigen-binding fragments thereof of the present invention or any of the pharmaceutical compositions of the present invention.

In some embodiments, the monoclonal antibody comprises three heavy chain complementarity-determining regions HCDR1, HCDR2, and HCDR3, and three light chain complementarity-determining regions LCDR1, LCDR2, and LCDR3, wherein: the amino acid sequence of the HCDR1 is set forth in SEQ ID NO: 1 or a variant thereof with one to three conservative amino acid substitutions; the amino acid sequence of the HCDR2 is set forth in SEQ ID NO: 2 or a variant thereof with one to three conservative amino acid substitutions; the amino acid sequence of the HCDR3 is set forth in SEQ ID NO: 6 or a variant thereof with one to three conservative amino acid substitutions; the amino acid sequence of the LCDR1 is set forth in SEQ ID NO: 7 or a variant thereof with one to three conservative amino acid substitutions; the amino acid sequence of the LCDR2 is set forth in SEQ ID NO: 9 or a variant thereof with one to three conservative amino acid substitutions; and the amino acid sequence of the LCDR3 is set forth in SEQ ID NO: 11 or a variant thereof with one to three conservative amino acid substitutions. In some embodiments, the amino acid sequence of the HCDR1 is set forth in SEQ ID NO: 1; the amino acid sequence of the HCDR2 is set forth in SEQ ID NO: 2, SEQ ID NO: 3, SEQ ID NO: 4, or SEQ ID NO: 5; the amino acid sequence of the HCDR3 is set forth in SEQ ID NO: 6; the amino acid sequence of the LCDR1 is set forth in SEQ ID NO: 7 or SEQ ID NO: 8; the amino acid sequence of the LCDR2 is set forth in SEQ ID NO: 9 or SEQ ID NO: 10; and the amino acid sequence of the LCDR3 is set forth in SEQ ID NO: 11.

In some embodiments, the monoclonal antibody comprises a heavy chain variable region (VH) and a light chain variable region (VL), wherein:
(a) the VH comprises the amino acid sequence set forth in SEQ ID NO: 12 or a variant having at least 85% sequence identity thereto; the VL comprises the amino acid sequence set forth in SEQ ID NO: 18 or a variant having at least 85% sequence identity thereto;
(b) the VH comprises the amino acid sequence set forth in SEQ ID NO: 12 or a variant having at least 85% sequence identity thereto; the VL comprises the amino acid sequence set forth in SEQ ID NO: 21 or a variant having at least 85% sequence identity thereto; or
(c) the VH comprises the amino acid sequence set forth in SEQ ID NO: 14 or a variant having at least 85% sequence identity thereto; the VL comprises the amino acid sequence set forth in SEQ ID NO: 18 or a variant having at least 85% sequence identity thereto.

In some embodiments, the monoclonal antibody comprises a heavy chain and a light chain, wherein:
(a) the heavy chain comprises the amino acid sequence set forth in SEQ ID NO: 28 or a variant having at least 85% sequence identity thereto; the light chain comprises the amino acid sequence set forth in SEQ ID NO: 34 or a variant having at least 85% sequence identity thereto;
(b) the heavy chain comprises the amino acid sequence set forth in SEQ ID NO: 28 or a variant having at least 85% sequence identity thereto; the light chain comprises the amino acid sequence set forth in SEQ ID NO: 35 or a variant having at least 85% sequence identity thereto;
(c) the heavy chain comprises the amino acid sequence set forth in SEQ ID NO: 29 or a variant having at least 85% sequence identity thereto; the light chain comprises the amino acid sequence set forth in SEQ ID NO: 34 or a variant having at least 85% sequence identity thereto;
(d) the heavy chain comprises the amino acid sequence set forth in SEQ ID NO: 30 or a variant having at least 85% sequence identity thereto; the light chain comprises the amino acid sequence set forth in SEQ ID NO: 34 or a variant having at least 85% sequence identity thereto;
(e) the heavy chain comprises the amino acid sequence set forth in SEQ ID NO: 30 or a variant having at least 85% sequence identity thereto; the light chain comprises the amino acid sequence set forth in SEQ ID NO: 35 or a variant having at least 85% sequence identity thereto; or
(f) the heavy chain comprises the amino acid sequence set forth in SEQ ID NO: 31 or a variant having at least 85% sequence identity thereto; the light chain comprises the amino acid sequence set forth in SEQ ID NO: 34 or a variant having at least 85% sequence identity thereto.

In some embodiments, the monoclonal antibody binds to CD38, wherein the monoclonal antibody binds to an extracellular domain of CD38 at amino acid residues comprising at least one amino acid residue selected from a group consisting of K25, E28, 129, M33, and R34, wherein the amino acid residue is numbered according to the amino acid sequence of the extracellular domain of CD38 as shown in SEQ ID NO: 36. In some embodiments, the monoclonal antibody binds to CD38, wherein the monoclonal antibody binds to an extracellular domain of CD38 at amino acid residues consisting of K25, E28, 129, P31, E32, M33, R34, H3, K67, T70, Q71, T72 and V73.

In some embodiments, the CD38-positive cancer of the present invention is a hematologic cancer or a solid tumor. In some embodiments, the hematologic cancer includes, but is not limited to, multiple myeloma (MM), leukemia (acute lymphocytic leukemia (ALL), acute myelogenous leukemia (AML), chronic lymphocytic leukemia (CLL), chronic myelogenous leukemia (CML), juvenile myelomonocytic leukemia (JML), adult T-cell lymphocytic leukemia (ATL), plasma cell leukemia), and lymphoma (small lymphocytic lymphoma, lymphoplasmacytic lymphoma, marginal zone lymphoma, follicular lymphoma, mantle cell lymphoma, diffuse large cell B-cell lymphoma, and Burkitt lymphoma). In some embodiments, the hematologic cancer is multiple myeloma (MM). In some embodiments, the hematologic cancer is relapsed/refractory multiple myeloma (R/R MM).

In some embodiments, the solid tumor includes, but is not limited to, melanoma, lung cancer, squamous non-small cell lung cancer (NSCLC), non-squamous NSCLC, colorectal cancer, prostate cancer, castration-resistant prostate cancer, gastric cancer, ovarian cancer, liver cancer, pancreatic cancer, thyroid cancer, head and neck squamous cell carcinoma, esophagus or gastrointestinal cancer, breast cancer, fallopian tube cancer, brain cancer, urethral cancer, genitourinary cancer, endometrial cancer, cervical cancer, lung adenocarcinoma, renal cell carcinoma (RCC) (e.g., kidney clear cell carcinoma or kidney papillary cell carcinoma), mesothelioma, nasopharyngeal carcinoma (NPC), esophagus cancer, or gastrointestinal cancer or a metastatic lesion of any of the foregoing. In some embodiments, the solid tumor is lung cancer.

In some embodiments, the autoimmune disease is selected from the group consisting of rheumatoid arthritis, systemic lupus erythematosus, multiple sclerosis, myasthenia gravis, type 1 diabetes mellitus, thrombocytopenia purpura, Sjogren's syndrome, Guillain-Barré syndrome, toxic diffuse goiter (Graves' disease), vasculitis, asthma, lymphoma, Alzheimer's disease, amyloid cardiomyopathy, warm autoimmune hemolytic anemia (wAIHA), colorectal carcinoma, immune thrombocytopenia, lupus nephritis, macroglobulinemia, hypergammaglobulinemia, autoimmune hemolytic anemia, and neuromyelitis optica. In some embodiments, the autoimmune disease is systemic lupus erythematosus, warm autoimmune hemolytic anemia, or immune thrombocytopenia.

### EXAMPLES

### Example 1. Hybridoma Preparation and Screening

A CD38 monoclonal antibody was prepared using hybridoma technology. A recombinant extracellular domain of CD38 was mixed with an equal volume of incomplete Freund's adjuvant, and the mixture was intramuscularly injected into a BALB/c mouse for immunization. After three rounds of primary immunization, blood was collected from the tail vein and the mouse serum was assayed for antibody titer using ELISA. A booster immunization was administered three days before fusion. First, the spleen of the immunized mouse was fused in batches with SP2/0 by following the experimental steps of hybridoma cell fusion. The fused cells were then resuspended in a HAT-containing medium, and the suspensions were transferred to 96-well plates. The plates were marked according to the reference numbers and incubated in a 37° C. cell incubator. The culture medium was changed regularly, and growth was monitored periodically. After 10⁻¹² days of culture, the supernatant was collected for ELISA testing. The recombinant extracellular domain of CD38 was purchased from AtaGenix Laboratories (Wuhan) Co., Ltd. under catalog No. ATMP00140HU, and its amino acid sequence is shown in the table below.

TABLE 1
The amino acid sequence of the recombinant extracellular domain
of CD38

| | |
|---|---|
| MKHLWFFLLLVAAPRWVLSVPRWRQQWSGPGTTKRFPETVLARCVKYTEIHPEMR | SEQ ID |
| HVDCQSVWDAFKGAFISKHPCNITEEDYQPLMKLGTQTVPCNKILLWSRIKDLAHQ | NO: 22 |
| FTQVQRDMFTLEDTLLGYLADDLTWCGEFNTSKINYQSCPDWRKDCSNNPVSVFW | |
| KTVSRRFAEAACDVVHVMLNGSRSKIFDKNSTFGSVEVHNLQPEKVQTLEAWVIHG | |
| GREDSRDLCQDPTIKELESIISKRNIQFSCKNIYRPDKFLQCVKNPEDSSCTSEIGSHHH | |
| HHH | |

The signal peptide is underlined, and the GS linker and His tag are italicized

Antibody clones capable of binding to CD38-positive cells were selected using ELISA and flow cytometry, and hybridoma cells capable of binding to the extracellular domain of CD38 with high affinity were finally selected using SPR technology. The ability of the selected hybridomas to induce apoptosis was tested with an Annexin V/PI apoptosis assay kit, and hybridoma H2 was selected.

As shown in FIG. 1, H2 exhibited apoptotic activity on all the four types of CD38-positive cells: Daudi, Ramos, Raji, and SU-DHL-1; the apoptotic activity of H2 on Daudi, Ramos, and Raji cells was significantly superior to that of the positive control Dara, and the apoptotic activity of H2 on SU-DHL-1 cells was comparable to that of the positive control Dara. Biacore affinity testing results (Table 2) indicate that the affinity constant of the H2 antibody was comparable to that of the positive control Dara, suggesting that it has a comparable binding affinity to Dara. The H2 mAb was sequenced, and the sequences are shown in Table 3 below.

TABLE 2
Affinity testing of the H2 mAb

| Sample name | KD (M) | Ka (1/Ms) | Kd (1/s) |
|---|---|---|---|
| Dara | 1.22 × 10⁻⁸ | 2.97 × 10⁵ | 3.63 × 10⁻³ |
| H2 | 1.46 × 10⁻⁸ | 6.61 × 10⁴ | 9.69 × 10⁻⁴ |

TABLE 3
The complementarity-determining region sequences and variable
region sequences of the H2 antibody

| Sequence | | SEQ ID |
|---|---|---|
| name | Sequence | NO: # |
| HCDR1 | DYNVH | 1 |
| HCDR2 | YFYPRNGATHYNQKFTG | 2 |
| HCDR3 | GETPGTFPY | 6 |
| LCDR1 | RASESVDNFGITFMH | 7 |
| LCDR2 | RASNLES | 9 |
| LCDR3 | QQSSKDPRT | 11 |
| VH | EVQLQESGAELVRSGASVKMSCKASGYTFTDYNVHWIKQTPGQG | 23 |
| | LEWIGYFYPRNGATHYNQKFTGKATLTADTSSSTAYIQISSLTSEDS | |
| | AVYFCARGETPGTFPYWGQGTLVTVSA | |
| VL | DIVLTQSPASLTVSLGQRATISCRASESVDNFGITFMHWYQQKPGQP | 24 |
| | PKLLIYRASNLESGIPARFSGSGSRTDFTLTIDPVETDDVATYYCQQ | |
| | SSKDPRTFGGGTKLEIK | |

### Example 2. Elisa Assay for Competitive Occupancy of Antigenic

### Epitopes

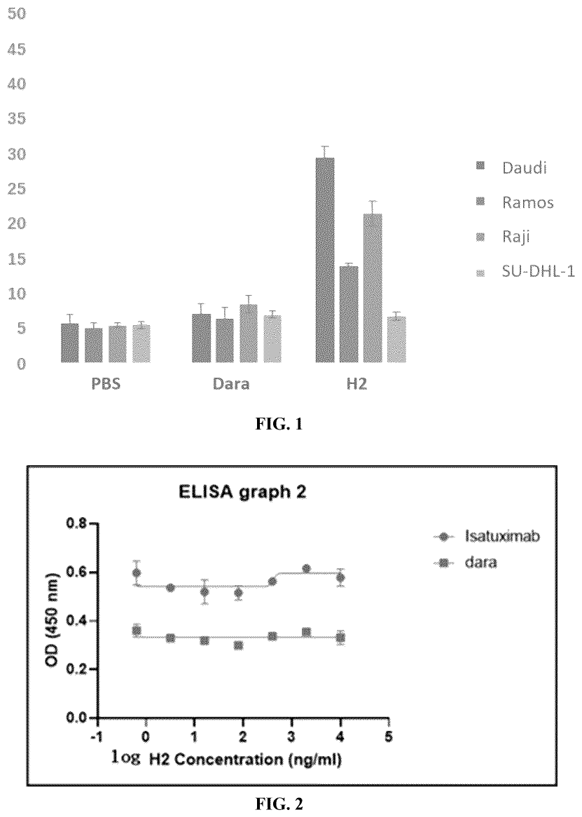

After coating with the antigen CD38, different concentrations of the H2 mAb were added and incubated thoroughly. Then Isa and Dara were added in a saturation concentration or more, and the binding of Isa and Dara was measured. If the test antibody's binding site on the CD38 protein obstructs or overlaps that of Isa or Dara to some extent, the binding activity of Isa or Dara will be reduced. The results indicate that the H2 antibody bound to a different antigenic epitope than Isa or Dara did (FIG. 2).

### Example 3. Cross-Reactivity of CD38 Antibodies Between Antigens of

### Different Species

The cross-reactivity of the antibodies between CD38 antigen proteins of different species was measured using Elisa. Coating was performed with CD38 antigens of different species (human, cynomolgus, mouse, and rat) (1 μg/mL, 50 mL/well), and incubation was performed overnight at 2-8° C. Antibody concentrations: 300, 100, 33.3, 11.1, 3.7, 1.23, and 0.41 g/mL.

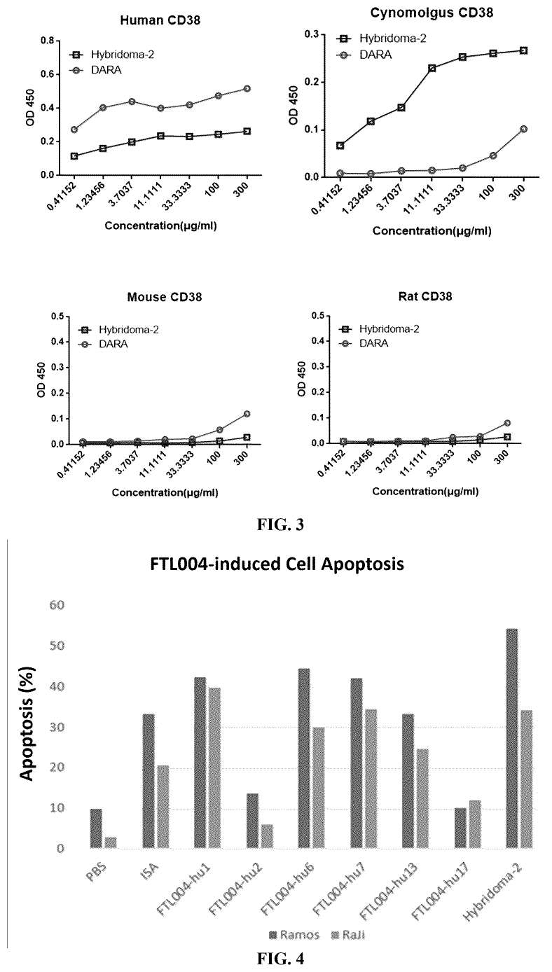

Elisa results show that (FIG. 3): the Dara antibody bound to the human antigen and did not bind to the cynomolgus, mouse, and rat antigens; the H2 antibody (Hybridoma-2) bound to the human and monkey antigens and did not bind to the mouse and rat antigens.

### Example 4. H2 Antibody Humanization, Chimeric Antibody Design, Vector Construction and Expression

First, sequences of a murine antibody were used to search for top-ranking sequences within a human framework library as candidate sequences. Then, the human sequences are homology-modeled onto the backbone of the murine antibody, with the sequences of the murine CDRs kept. Then the energy of the homology model was calculated to assess the stability of the humanized antibody. For humanized CDR mutants, the same strategy was applied, except that the murine CDR sequences were replaced with mutant sequences and the structure of the antigen was retained. According to the humanization design and engineering, 18 humanized sequences (denoted by FLT004-hu-1 to FLT004-hu-18; the specific sequences are shown in Tables 4 to 6 below) were designed from the parent mAb H2. According to the designed humanized sequences, the target sequences were constructed and transiently transfected into HEK293E cells, and chimeric antibodies were expressed.

TABLE 4
The CDR sequences of humanized antibodies

| Antibody | HCDR1 | HCDR2 | HCDR3 | LCDR1 | LCDR2 | LCDR3 |
|---|---|---|---|---|---|---|
| FTL004-hu-1/3/5/7/ | DYN | YFYPRNGATHYN | GETPG | RASESVDNFGI | RASNLE | QQSSK |
| 9/11/13/15/17 | VH | QKFTG | TFPY | TFMH | S | DPRT |
| FTL004-hu-2/4 | DYN | YFYPRNGATHYA | GETPG | RASESVDNFGI | RASNLE | QQSSK |
| | VH | QGFTG(SEQ ID | TFPY | TFMH | S | DPRT |
| | | NO: 3) | | | | |
| FTL004-hu-6/12/18 | DYN | YFYPRNGATHYN | GETPG | QASESVDNFGI | RASNLE | QQSSK |
| | VH | QKFTG | TFPY | TFMH(SEQ ID | T(SEQ ID | DPRT |
| | | | | NO: 8) | NO: 10) | |
| FTL004-hu-8/10 | DYN | YFYPRNGATHYA | GETPG | RASESVDNFGI | RASNLE | QQSSK |
| | VH | QKFQG(SEQ ID | TFPY | TFMH | S | DPRT |
| | | NO: 4) | | | | |
| FTL004-hu-14/16 | DYN | YFYPRNGATHYA | GETPG | RASESVDNFGI | RASNLE | QQSSK |
| | VH | EKFQG(SEQ ID | TFPY | TFMH | S | DPRT |
| | | NO: 5) | | | | |

TABLE 5
The variable region sequences of humanized antibodies

| | | SEQ ID |
|---|---|---|
| Antibody | VH | NO: # |
| FTL004-hu-1/3/5/ | QVQLVQSGSELKKPGASVKVSCKASGYTFTDYNVHWIRQAPG | 12 |
| 6 | QGLEWIGYFYPRNGATHYNQKFTGRAVLSADTSVSTAYLQISSL | |
| | KAEDTAVYFCARGETPGTFPYWGQGTLVTVSS | |
| FTL004-hu-2/4 | QVQLVQSGSELKKPGASVKVSCKASGYTFTDYNVHWIRQAPG | 13 |
| | QGLEWIGYFYPRNGATHYAQGFTGRAVLSADTSVSTAYLQISSL | |
| | KAEDTAVYYCARGETPGTFPYWGQGTLVTVSS | |
| FTL004-hu-7/9/1 | QVQLVQSGAEVKKPGASVKVSCKASGYTFTDYNVHWIRQAPG | 14 |
| 1/12 | QGLEWIGYFYPRNGATHYNQKFTGRATLTVDTSTSTAYMEISSL | |
| | RSEDTAVYFCARGETPGTFPYWGQGTLVTVSS | |
| FTL004-hu-8/10 | QVQLVQSGAEVKKPGASVKVSCKASGYTFTDYNVHWIRQAPG | 15 |
| | QGLEWIGYFYPRNGATHYAQKFQGRVTMTVDTSTSTAYMELSS | |
| | LRSEDTAVYYCARGETPGTFPYWGQGTLVTVSS | |
| FTL004-hu-13/15/ | EVQLVQSGAEVKKPGATVKISCKASGYTFTDYNVHWIQQAPG | 16 |
| 17/18 | KGLEWIGYFYPRNGATHYNQKFTGRATLTADTSTDTAYMEISSL | |
| | RSEDTAVYFCARGETPGTFPYWGQGTLVTVSS | |
| FTL004-hu-14/16 | EVQLVQSGAEVKKPGATVKISCKASGYTFTDYNVHWIQQAPG | 17 |
| | KGLEWIGYFYPRNGATHYAEKFQGRVTLTADTSTDTAYMELSS | |
| | LRSEDTAVYYCARGETPGTFPYWGQGTLVTVSS | |
| | | SEQ ID |
| Antibody | VL | NO: # |
| FTL004-hu-1/2/7/ | DIVLTQSPASLAVSPGQRATITCRASESVDNFGITFMHWYQQKP | 18 |
| 8/13/14 | GQPPKLLIYRASNLESGVPARFSGSGSRTDFTLTINPVEANDTAN | |
| | YYCQQSSKDPRTFGQGTKLEIK | |
| FTL004-hu-3/4/9/ | EIVLTQSPATLSLSPGERATLSCRASESVDNFGITFMHWYQQKPG | 19 |
| 10/15/16 | QAPRLLIYRASNLESGIPARFSGSGSRTDFTLTISSLEPEDFAVYY | |
| | CQQSSKDPRTFGQGTKLEIK | |
| FTL004-hu-5/11/ | DIQLTQSPSSLSASVGDRVTITCRASESVDNFGITFMHWYQQKP | 20 |
| 17 | GKAPKLLIYRASNLESGVPSRFSGSGSRTDFTLTISSLQPEDFATY | |
| | YCQQSSKDPRTFGQGTKLEIK | |
| FTL004-hu-6/12/ | DIQLTQSPSSLSASVGDRVTITCQASESVDNFGITFMHWYQQKP | 21 |
| 18 | GKAPKLLIYRASNLETGVPSRFSGSGSRTDFTFTISSLQPEDIATY | |
| | YCQQSSKDPRTFGQGTKLEIK | |

TABLE 6
The constant region sequences of humanized antibodies

| | | SEQ ID |
|---|---|---|
| Name | Sequence | NO: # |
| Heavy chain | ASTKGPSVFPLAPSSKSTSGGTAALGCLVKDYFPEPVTVSWN | 25 |
| constant regions | SGALTSGVHTFPAVLQSSGLYSLSSVVTVPSSSLGTQTYICNV | |
| (CH) of | NHKPSNTKVDKKVEPKSCDKTHTCPPCPAPELLGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| FTL004-hu-1 to −18 | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Light chain constant | RTVAAPSVFIFPPSDEQLKSGTASVVCLLNNFYPREAKVQWK | 33 |
| regions (CL) of | VDNALQSGNSQESVTEQDSKDSTYSLSSTLTLSKADYEKHK | |
| FTL004-hu-1 to −18 | VYACEVTHQGLSSPVTKSFNRGEC | |

### Example 5. Biacore Affinity Assay of Humanized Antibodies

The affinities of the humanized antibodies were measured by Biacore, and the results are shown in Table 7 below. The affinity constants of FTL004-hu-1/3/5/6/7/11/13 were comparable to that of the positive control Dara, suggesting that they have comparable binding affinities to Dara.

TABLE 7
[]Affinity analysis of humanized antibodies

| | Kinetics | | | | |
|---|---|---|---|---|---|
| | Chi² | | | | Rmax |
| Analyte | (RU²) | ka (1/Ms) | kd (1/s) | KD (M) | (RU) |
| Dara (positive | 2.72E-01 | 1.81E+05 | 3.55E-04 | 1.96E-09 | 51.4 |
| control | | | | | |
| antibody) | | | | | |
| Isa (positive | 1.36E+00 | 2.90E+05 | 2.77E-04 | 9.56E-10 | 58.0 |
| control | | | | | |
| antibody) | | | | | |
| H2 | 1.08E+00 | 2.48E+05 | 5.00E-04 | 2.02E-09 | 57.0 |
| FTL004-hu-1 | 5.24E+00 | 5.35E+05 | 7.59E-04 | 1.42E-09 | 79.9 |
| FTL004-hu-2 | 1.17E+00 | 3.44E+05 | 2.85E-03 | 8.30E-09 | 52.8 |
| FTL004-hu-3 | 2.73E+00 | 3.47E+05 | 5.20E-04 | 1.50E-09 | 78.5 |
| FTL004-hu-4 | 7.30E-01 | 2.97E+05 | 3.13E-03 | 1.05E-08 | 45.2 |
| FTL004-hu-5 | 3.98E+00 | 4.15E+05 | 6.86E-04 | 1.65E-09 | 75.9 |
| FTL004-hu-6 | 1.49E+00 | 2.77E+05 | 6.77E-04 | 2.44E-09 | 66.1 |
| FTL004-hu-7 | 1.91E+00 | 3.20E+05 | 7.66E-04 | 2.40E-09 | 70.4 |
| FTL004-hu-8 | 1.10E+00 | 3.07E+05 | 2.57E-03 | 8.39E-09 | 50.4 |
| FTL004-hu-9 | 8.21E-01 | 2.30E+05 | 6.99E-04 | 3.04E-09 | 63.4 |
| FTL004-hu-10 | 3.26E-01 | 1.62E+05 | 2.41E-03 | 1.49E-08 | 49.0 |
| FTL004-hu-11 | 1.26E+00 | 2.96E+05 | 8.48E-04 | 2.87E-09 | 62.6 |
| FTL004-hu-12 | 8.77E-01 | 2.34E+05 | 8.30E-04 | 3.54E-09 | 60.8 |
| FTL004-hu-13 | 1.58E+00 | 3.07E+05 | 8.53E-04 | 2.78E-09 | 61.2 |
| FTL004-hu-14 | 2.21E-01 | 2.08E+05 | 4.66E-03 | 2.24E-08 | 36.6 |
| FTL004-hu-15 | 5.98E-01 | 2.14E+05 | 7.84E-04 | 3.66E-09 | 56.1 |
| FTL004-hu-16 | 1.88E-01 | 1.82E+05 | 4.59E-03 | 2.52E-08 | 35.4 |
| FTL004-hu-17 | 1.21E+00 | 2.75E+05 | 8.48E-04 | 3.09E-09 | 194.9 |
| FTL004-hu-18 | 3.51E-01 | 1.89E+05 | 7.88E-04 | 4.16E-09 | 119.8 |

### Example 6. Apoptotic Activity Assay of Humanized Antibodies

The apoptotic activity of the humanized antibodies was measured, and some of the results are shown in FIG. 4. The results show that FTL004-hu-1/6/7 have the best apoptosis-inducing activity and exhibited better apoptosis-inducing activity than the positive control Isa on both Ramos and Raji cells; FTL004-hu-13 has the second best apoptotic activity and exhibited comparable apoptosis-inducing activity to the positive control Isa on Ramos cells, and better apoptosis-inducing activity than the positive control Isa on Raji cells.

The activity of the humanized antibodies was measured by ELISA, and the results are shown in FIG. 5. Overall comparison of absorbance curves and EC50 values show that FTL004-hu-1/3/5/6/7/9 (corresponding to humanized-1/3/5/6/7/9 in the figure, respectively) have relatively good activity, all surpassing the positive control Isa; the activity of FTL004-hu-11/12/13/15/17 (corresponding to humanized-11/12/13/15/17 in the figure, respectively) was second to that of FTL004-hu-1/3/5/6/7/9.

### Example 7. Antibody Stability Testing

High-temperature stability: After humanized antibody samples were left to stand at −20° C. and 40° C. for one week, the percentages of aggregates, monomers, and fragments in the samples were analyzed, and the results are shown in the table below. For some antibodies, no significant change in SEC purity was observed after one week of standing at 40° C. According to analysis of the initial purity of the products, the products FTL004-hu-1/2/5/6/7 (corresponding to FTL004-hum-1/2/5/6/7 in Table 8) were believed to have both relatively good initial purity and stability. They may have advantages in light and heavy chain expression, assembly, and secretion within cells. The results are shown in Table 8.

TABLE 8
High-temperature stability analysis of humanized antibodies

| Standing conditions: −20° C., 1 week | | | | Standing conditions: 40° C., 60% humidity, 1 week | | | |
|---|---|---|---|---|---|---|---|
| | Aggregate | Monomer | Fragment | | Aggregate | Monomer | Fragment |
| Sample name | (%) | (%) | (%) | Sample name | (%) | (%) | (%) |
| FTL004-hum-1 | 0.5556 | 99.4444 | 0 | FTL004-hum-1 40° C. 1 week | 0.5363 | 99.4637 | 0 |
| FTL004-hum-2 | 5.3982 | 94.6018 | 0 | FTL004-hum-2 40° C. 1 week | 4.3961 | 95.6039 | 0 |
| FTL004-hum-3 | 11.4135 | 88.5865 | 0 | FTL004-hum-3 40° C. 1 week | 11.7626 | 88.1364 | 0.101 |
| FTL004-hum-4 | 16.7801 | 82.767 | 0.4529 | FTL004-hum-4 40° C. 1 week | 19.7386 | 80.2614 | 0 |
| FTL004-hum-5 | 1.9175 | 97.9548 | 0.1277 | FTL004-hum-5 40° C. 1 week | 2.0852 | 97.6047 | 0.3101 |
| FTL004-hum-6 | 4.9803 | 95.0197 | 0 | FTL004-hum-6 40° C. 1 week | 4.6457 | 95.2196 | 0.1347 |
| FTL004-hum-7 | 9.6863 | 90.3137 | 0 | FTL004-hum-7 40° C. 1 week | 8.9183 | 91.0817 | 0 |
| FTL004-hum-8 | 19.5902 | 80.4098 | 0 | FTL004-hum-8 40° C. 1 week | 18.824 | 80.9483 | 0.2277 |
| FTL004-hum-9 | 21.404 | 76.6592 | 1.9368 | FTL004-hum-9 40° C. 1 week | 20.8592 | 78.7487 | 0.3921 |
| FTL004-hum-10 | 26.7826 | 48.5972 | 24.6202 | FTL004-hum-10 40° C. 1 week | 26.3701 | 48.1073 | 25.5226 |
| FTL004-hum-11 | 21.917 | 78.083 | 0 | FTL004-hum-11 40° C. 1 week | 21.9722 | 77.9274 | 0.1004 |
| FTL004-hum-12 | 27.0415 | 72.3258 | 0.6327 | FTL004-hum-12 40° C. 1 week | 28.77 | 71.23 | 0 |
| FTL004-hum-13 | 12.3497 | 87.6503 | 0 | FTL004-hum-13 40° C. 1 week | 12.5252 | 87.4748 | 0 |
| FTL004-hum-14 | 30.2723 | 69.7277 | 0 | FTL004-hum-14 40° C. 1 week | 30.0644 | 69.9356 | 0 |
| FTL004-hum-15 | 37.4911 | 62.5089 | 0 | FTL004-hum-15 40° C. 1 week | 36.7698 | 63.2302 | 0 |
| FTL004-hum-16 | 36.4911 | 43.1392 | 20.3697 | FTL004-hum-16 40° C. 1 week | 36.1959 | 42.7672 | 21.0369 |
| FTL004-hum-17 | 27.877 | 72.123 | 0 | FTL004-hum-17 40° C. 1 week | 27.9009 | 72.0991 | 0 |
| FTL004-hum-18 | 27.3342 | 72.6658 | 0 | FTL004-hum-18 40° C. 1 week | 30.3506 | 69.4121 | 0.2373 |

Oxidation stability: several samples with relatively good activity were selected for oxidation stability testing, and the results are shown in the table below. It can be seen that both 6 h and 24 h of treatment with 0.1% H₂O₂ resulted in increases in SEC fragment peak content. The results are shown in Table 9. Overall comparison: The antibodies FTL004-hu-1/2/6/7/13 have the best oxidation stability, with relatively small increases in fragment peak content; the antibodies FTL004-hu-5//9/11/15/17 have relatively poor stability, with relatively big increases in fragment peak content.

TABLE 9
Oxidation stability analysis of humanized antibodies

| Standing conditions: H₂O₂-free | | | | Standing conditions: 0.1% H₂O₂, 6 h | | | | Standing conditions: 0.1% H₂O₂, 24 h | | | |
|---|---|---|---|---|---|---|---|---|---|---|---|
| | Aggre- | | | | Aggre- | | | | Aggre- | | |
| Sample | gate | Monomer | Fragment | | gate | Monomer | Fragment | | gate | Monomer | Fragment |
| name | (%) | (%) | (%) | Sample name | (%) | (%) | (%) | Sample name | (%) | (%) | (%) |
| FTL004- | 0.5556 | 99.4444 | 0 | FTL004-hu-1- | 0.8784 | 98.5132 | 0.6084 | FTL004-hu-1- | 0.3811 | 98.6225 | 0.9964 |
| hu-1 | | | | 0.1% HP-6 h | | | | 0.1% HP-24 h | | | |
| FTL004- | 11.4135 | 88.5865 | 0 | FTL004-hu-3- | 10.7138 | 88.4666 | 0.8196 | FTL004-hu-3- | 9.6561 | 88.5127 | 1.8012 |
| hu-3 | | | | 0.1% HP-6 h | | | | 0.1% HP-24 h | | | |
| FTL004- | 1.9175 | 97.9548 | 0.1277 | FTL004-hu-5- | 2.0761 | 96.6181 | 1.3058 | FTL004-hu-5- | 1.668 | 94.1033 | 4.2287 |
| hu-5 | | | | 0.1% HP-6 h | | | | 0.1% HP-24 h | | | |
| FTL004- | 4.9603 | 95.0197 | 0 | FTL004-hu-6- | 4.6211 | 94.2992 | 1.0797 | FTL004-hu-6- | 4.2977 | 93.62 | 2.0823 |
| hu-6 | | | | 0.1% HP-6 h | | | | 0.1% HP-24 h | | | |
| FTL004- | 9.6863 | 90.3137 | 0 | FTL004-hu-7- | 8.9149 | 89.9815 | 1.1036 | FTL004-hu-7- | 7.864 | 90.3831 | 1.7529 |
| hu-7 | | | | 0.1% HP-6 h | | | | 0.1% HP-24 h | | | |
| FTL004- | 21.404 | 76.6592 | 1.9368 | FTL004-hu-9- | 13.6459 | 76.5251 | 9.829 | FTL004-hu-9- | 14.0501 | 76.0095 | 9.9404 |
| hu-9 | | | | 0.1% HP-6 h | | | | 0.1% HP-24 h | | | |
| FTL004- | 21.917 | 78.063 | 0 | FTL004-hu-11- | 17.3898 | 76.9046 | 5.7056 | FTL004-hu-11- | 16.7853 | 77.0061 | 6.2086 |
| hu-11 | | | | 0.1% HP-6 h | | | | 0.1% HP-24 h | | | |
| FTL004- | 12.3497 | 87.6503 | 0 | FTL004-hu-13- | 11.0739 | 87.6065 | 1.3176 | FTL004-hu-13- | 9.9312 | 88.1892 | 1.8796 |
| hu-13 | | | | 0.1% HP-6 h | | | | 0.1% HP-24 h | | | |
| FTL004- | 37.4911 | 62.5069 | 0 | FTL004-hu-15- | 31.1343 | 64.8729 | 3.9928 | FTL004-hu-15- | 30.2768 | 64.9916 | 4.7316 |
| hu-15 | | | | 0.1% HP-6 h | | | | 0.1% HP-24 h | | | |
| FTL004- | 27.877 | 72.123 | 0 | FTL004-hu-17- | 23.5579 | 72.7951 | 1.647 | FTL004-hu-17- | 20.4142 | 71.6786 | 7.9072 |
| hu-17 | | | | 0.1% HP-6 h | | | | 0.1% HP-24 h | | | |

From the above results, the antibodies FTL004-hu-1/6/7 were preliminarily believed to have relatively good physicochemical properties. The overall stability of the three was tested (the results are shown in Table 10 below). The humanized antibody No. 1 exhibited the best stability, was sensitive to oxidation, and was relatively stable under high-temperature, acidic-alkaline, freeze-thaw, etc. conditions.

TABLE 10
Overall stability analysis of humanized antibodies

| | | FTL004-hu-1 | | | | | | FTL004-hu-6 | | |
|---|---|---|---|---|---|---|---|---|---|---|
| | | SEC | | | IEC | | | SEC | | |
| | | Aggre- | Main | Frag- | Acid | Main | Alkali | Aggre- | Main | Frag- |
| Experimental | | gate | Peak | ment | Peak | Peak | peak | gate | Peak | ment |
| conditions | | (%) | (%) | (%) | (%) | (%) | (%) | (%) | (%) | (%) |
| N/A | Starting | 2.01 | 97.99 | 0.00 | 43.18 | 54.04 | 2.78 | 5.94 | 94.06 | 0.00 |
| | sample | | | | | | | | | |
| Low pH | pH 3.4 1 h | 2.15 | 97.85 | 0.00 | 43.76 | 53.25 | 3.00 | 5.84 | 94.16 | 0.00 |
| | pH 3.4 3 h | 2.30 | 97.70 | 0.00 | 43.96 | 53.03 | 3.01 | 5.84 | 94.16 | 0.00 |
| | pH 4.0 5 h | 1.87 | 98.13 | 0.00 | 43.23 | 54.16 | 2.61 | 5.73 | 94.06 | 0.21 |
| | pH 4.0 20 h | 1.63 | 98.38 | 0.00 | 43.28 | 54.17 | 2.56 | 5.66 | 94.34 | 0.00 |
| | pH 4.0 44 h | 1.64 | 98.36 | 0.00 | 43.09 | 54.62 | 2.29 | 5.59 | 94.41 | 0.00 |
| High pH | pH 10.0 5 h | 2.31 | 97.52 | 0.17 | 43.84 | 53.55 | 2.61 | 6.26 | 93.74 | 0.00 |
| | pH 10.0 20 h | 2.04 | 97.83 | 0.13 | 44.17 | 53.32 | 2.51 | 6.32 | 93.68 | 0.00 |
| | pH 10.0 44 h | 1.87 | 97.94 | 0.18 | 44.91 | 52.93 | 2.16 | 6.42 | 93.58 | 0.00 |
| Oxidation | 0.1% H2O2 | 3.79 | 94.05 | 2.16 | 48.32 | 49.04 | 2.64 | 5.90 | 91.45 | 2.64 |
| | 20 h | | | | | | | | | |
| | 0.1% H2O2 | 3.89 | 91.49 | 4.62 | 51.05 | 46.38 | 2.57 | 6.65 | 89.65 | 3.70 |
| | 48 h | | | | | | | | | |
| | 0.1% H2O2 | 3.64 | 93.84 | 2.51 | 51.41 | 45.99 | 2.60 | 6.45 | 86.50 | 7.05 |
| | 70 h | | | | | | | | | |
| Freeze-thaw | 3 freeze-thaw | 1.93 | 98.07 | 0.00 | 43.36 | 53.41 | 3.23 | N/A | N/A | N/A |
| | cycles | | | | | | | | | |
| | 5 freeze-thaw | 1.81 | 98.19 | 0.00 | 43.43 | 53.38 | 3.19 | 5.75 | 94.25 | 0.00 |
| | cycles | | | | | | | | | |
| High | 40° C. 1 Week | 1.72 | 98.28 | 0.00 | 44.32 | 52.81 | 2.88 | 5.74 | 94.26 | 0.00 |
| temperature | 40° C. 2 Week | 2.27 | 97.54 | 0.19 | 45.97 | 51.42 | 2.61 | 5.82 | 94.04 | 0.14 |

| | | FTL004-hu-6 | | | FTL004-hu-7 | | | | | |
|---|---|---|---|---|---|---|---|---|---|---|
| | | IEC | | | SEC | | | IEC | | |
| | | Acid | Main | Alkali | Aggre- | Main | Frag- | Acid | Main | Alkali |
| Experimental | | Peak | Peak | peak | gate | Peak | ment | Peak | Peak | peak |
| conditions | | (%) | (%) | (%) | (%) | (%) | (%) | (%) | (%) | (%) |
| N/A | Starting | 7.34 | 78.44 | 14.22 | 9.38 | 90.62 | 0.00 | 41.82 | 55.83 | 2.35 |
| | sample | | | | | | | | | |
| Low pH | pH 3.4 1 h | 12.39 | 73.51 | 14.10 | 9.08 | 90.92 | 0.00 | 42.14 | 54.49 | 3.38 |
| | pH 3.4 3 h | 10.56 | 74.49 | 14.96 | 8.89 | 91.11 | 0.00 | 42.03 | 54.96 | 3.01 |
| | pH 4.0 5 h | 7.31 | 78.46 | 14.23 | 8.28 | 91.72 | 0.00 | 42.94 | 55.01 | 2.05 |
| | pH 4.0 20 h | 7.36 | 78.05 | 14.60 | 9.06 | 90.94 | 0.00 | 41.27 | 55.55 | 3.18 |
| | pH 4.0 44 h | 7.52 | 77.37 | 15.11 | 9.11 | 90.89 | 0.00 | 41.38 | 55.80 | 2.82 |
| High pH | pH 10.0 5 h | 8.02 | 77.94 | 14.04 | 9.10 | 90.90 | 0.00 | 41.84 | 56.14 | 2.02 |
| | pH 10.0 20 h | 9.27 | 76.82 | 13.92 | 8.14 | 91.86 | 0.00 | 47.65 | 50.70 | 1.65 |
| | pH 10.0 44 h | 11.12 | 75.69 | 13.20 | 8.13 | 91.87 | 0.00 | 51.84 | 46.71 | 1.45 |
| Oxidation | 0.1% H2O2 | 22.15 | 75.58 | 2.26 | 9.93 | 88.43 | 1.64 | 47.56 | 51.07 | 1.37 |
| | 20 h | | | | | | | | | |
| | 0.1% H2O2 | 25.58 | 72.28 | 2.14 | 9.75 | 87.80 | 2.45 | 49.07 | 49.55 | 1.39 |
| | 48 h | | | | | | | | | |
| | 0.1% H2O2 | 26.63 | 71.35 | 2.02 | 9.53 | 88.21 | 2.26 | 49.77 | 48.80 | 1.43 |
| | 70 h | | | | | | | | | |
| Freeze-thaw | 3 freeze-thaw | N/A | N/A | N/A | 9.32 | 90.68 | 0.00 | 41.54 | 55.79 | 2.67 |
| | cycles | | | | | | | | | |
| | 5 freeze-thaw | 11.83 | 73.40 | 14.77 | 9.40 | 90.60 | 0.00 | 42.04 | 55.31 | 2.65 |
| | cycles | | | | | | | | | |
| High | 40° C. 1 Week | 15.67 | 70.03 | 14.30 | 9.19 | 90.81 | 0.00 | 48.63 | 48.92 | 2.45 |
| temperature | 40° C. 2 Week | 20.31 | 66.26 | 13.43 | 5.84 | 94.16 | 0.00 | 55.26 | 42.49 | 2.25 |

In summary, the antibodies FTL004-hu-1/6/7 were believed to have relatively good activity and physicochemical properties. The three were subjected to subsequent Fc-mutation engineering.

### Example 8. In Vivo Pharmacodynamics of Humanized Antibodies

According to the previous screening results, the three humanized antibodies FTL004-hu-1/6/7, which have high affinities, good activity, and good stability, were selected for further animal pharmacodynamic testing. Animals: 3-5 NSG mice per group, male; cell line: SU-DHL-6, subcutaneous graft (1×10⁷ cells/0.2 mL/mouse). Dosage and frequency: 0, 2.5, 10 mg/kg; Q2Wx8 (the low-dose group was dosed 6 times); indicators: tumor volume, tumor weight, and the weight of the liver and kidney organs.

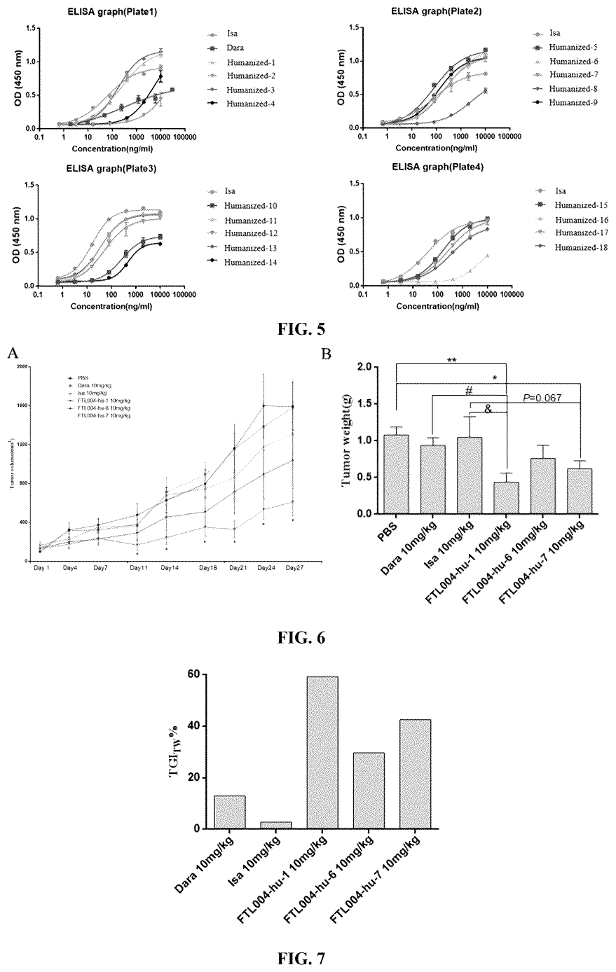

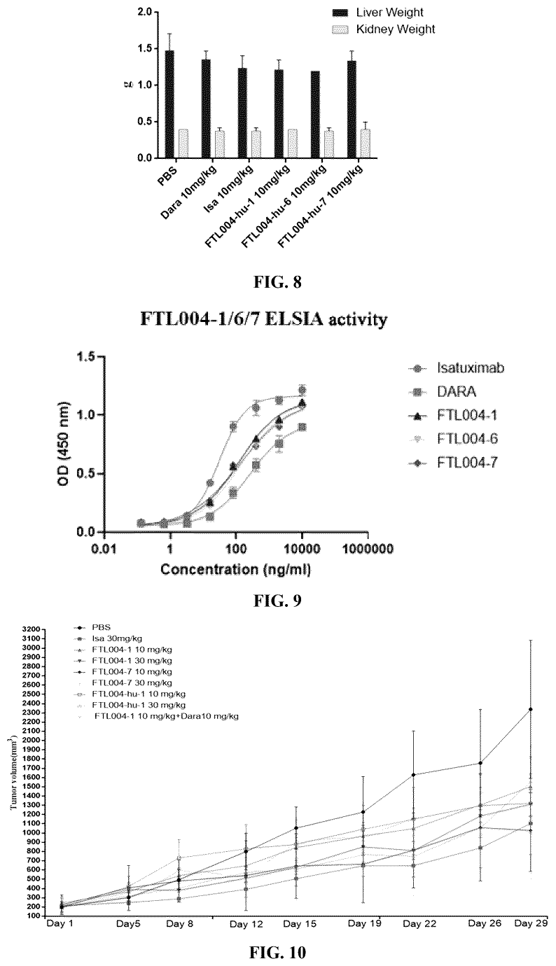

From the tumor growth curves (FIG. 6), it can be seen that: the intraperitoneal injections of 10 mg/kg FTL004-hu-1 (Q2Wx8) significantly inhibited tumor growth in SU-DHL-6 tumor-bearing mice, and the effect was significantly superior to that of the same dose of Dara and was superior to that of the same dose of Isa; although the tumor-inhibiting effect of 10 mg/kg FTL004-hu-6 and -7 was inferior to that of FTL004-hu-1, it was still superior to that of the same dose of Dara and Isa. At the end of the 27-day dosing experiment, tumors were dissected and weighed, and tumor inhibition rates were calculated (FIG. 7). For the experimental groups, the tumor inhibition rates are as follows: 12.96% for the Dara group; 2.78% for the Isa group; 59.26% for the FTL004-hu-1 group; 29.63% for FTL004-hu-6; and 42.59% for FTL004-hu-7. Thus, it can be seen that the tumor inhibition rates of FTL004-hu-1/6/7 were significantly superior to those of the positive controls Dara and Isa. Through general observations and weighing of the liver and kidney (FIG. 8), it was found that Dara, Isa, and FTL004-hu-1/6/7 all exhibited no significant hepatotoxicity or nephrotoxicity.

### Example 9. Engineering of Fc Portions of Antibodies

The Fc portion of an antibody interacts with many Fc receptors and ligands, producing a series of biological effects. For the Fc portion of an IgG, the Fc receptor family-FcγR plays a key role. The formation of Fc/FcγR complexes can effectively recruit corresponding effector cells, thereby leading to intracellular signaling and a series of important immune responses, such as the release of inflammatory mediators, B cell activation, encytosis, phagocytosis, and cytotoxic attacks. Through Fc portion engineering, antibody-dependent cell-mediated cytotoxicity (ADCC), antibody-dependent cell-mediated phagocytosis (ADCP), complement-dependent cytotoxicity (CDC), etc. can be enhanced.

The above-mentioned antibodies FTL004-hu-1/6/7 were subjected to Fc portion engineering using known methods in the art. The engineered antibodies FTL004-hu-1/6/7 were designated FTL004-1/6/7, respectively. In the Fc portions, the serine residue at position 19 was substituted with an aspartic acid residue (S19D), the isoleucine residue at position 112 was substituted with a glutamic acid residue (I112E), the aspartic acid residue at position 136 was substituted with a glutamic acid residue (D136E), the leucine residue at position 138 was substituted with a methionine residue (L138M), and the glycine and lysine residues at positions 226 and 227 were deleted. The engineered antibodies were designated FTL004-1/6/7. The N-termini of the antibody sequences further comprise a signal peptide set forth in SEQ ID NO: 32 (the specific sequence of which is shown in Table 12 below). Comparisons were made to look at the differences in activity and physicochemical properties between the antibodies before and after engineering.

TABLE 11
The sequences of the Fc portion before and after engineering

| Fc portion sequence before | Fc portion sequence after |
|---|---|
| engineering (SEQ ID NO: 26) | engineering (SEQ ID NO: 27) |
| DKTHTCPPCPAPELLGGPSVFLFPPKPKDTL | DKTHTCPPCPAPELLGGPDVFLFPPKPKDTL |
| MISRTPEVTCVVVDVSHEDPEVKFNWYVDG | MISRTPEVTCVVVDVSHEDPEVKFNWYVD |
| VEVHNAKTKPREEQYNSTYRVVSVLTVLHQ | GVEVHNAKTKPREEQYNSTYRVVSVLTVL |
| DWLNGKEYKCKVSNKALPAPIEKTISKAKG | HQDWLNGKEYKCKVSNKALPAPEEKTISK |
| QPREPQVYTLPPSRDELTKNQVSLTCLVKGF | AKGQPREPQVYTLPPSREEMTKNQVSLTCL |
| YPSDIAVEWESNGQPENNYKTTPPVLDSDGS | VKGFYPSDIAVEWESNGQPENNYKTTPPVL |
| FFLYSKLTVDKSRWQQGNVFSCSVMHEALH | DSDGSFFLYSKLTVDKSRWQQGNVFSCSV |
| NHYTQKSLSLSPGK | MHEALHNHYTQKSLSLSP |

TABLE 12
The sequence of the signal peptide

| | |
|---|---|
| MDPKGSLSWRILLFLSLAFELSYG | SEQ ID NO: 32 |

TABLE 13
The sequences of humanized antibodies before and after Fc
portion engineering

| | | SEQ ID |
|---|---|---|
| Antibody | Heavy chain | NO: # |
| FTL004-h | QVOLVOSGSELKKPGASVKVSCKASGYTFTDYNVHWIRQAPGOGLEWI | 28 |
| u-1/6 | GYFYPRNGATHYNQKFTGRAVLSADTSVSTAYLQISSLKAEDTAVYFCA | |
| | RGETPGTFPYWGQGTLVTVSSASTKGPSVFPLAPSSKSTSGGTAALGCL | |
| | VKDYFPEPVTVSWNSGALTSGVHTFPAVLQSSGLYSLSSVVTVPSSSLGT | |
| | QTYICNVNHKPSNTKVDKKVEPKSCDKTHTCPPCPAPELLGGPSVFLFPP | |
| | KPKDTLMISRTPEVTCVVVDVSHEDPEVKFNWYVDGVEVHNAKTKPREEQ | |
| | YNSTYRVVSVLTVLHODWLNGKEYKCKVSNKALPAPIEKTISKAKGQPREPQ | |
| | VYTLPPSRDELTKNQVSLTCLVKGFYPSDIAVEWESNGQPENNYKTTPPVLD | |
| | SDGSFFLYSKLTVDKSRWQQGNVFSCSVMHEALHNHYTOKSLSLSPGK | |
| FTL004-1/ | QVOLVOSGSELKKPGASVKVSCKASGYTFTDYNVHWIRQAPGOGLEWI | 30 |
| 6 | GYFYPRNGATHYNQKFTGRAVLSADTSVSTAYLQISSLKAEDTAVYFCA | |
| | RGETPGTFPYWGQGTLVTVSSASTKGPSVFPLAPSSKSTSGGTAALGCL | |
| | VKDYFPEPVTVSWNSGALTSGVHTFPAVLQSSGLYSLSSVVTVPSSSLGT | |
| | QTYICNVNHKPSNTKVDKKVEPKSCDKTHTCPPCPAPELLGGPDVFLFP | |
| | PKPKDTLMISRTPEVTCVVVDVSHEDPEVKFNWYVDGVEVHNAKTKPREE | |
| | QYNSTYRVVSVLTVLHQDWLNGKEYKCKVSNKALPAPEEKTISKAKGQPREP | |
| | QVYTLPPSREEMTKNQVSLTCLVKGFYPSDIAVEWESNGQPENNYKTTPPVL | |
| | DSDGSFFLYSKLTVDKSRWQQGNVFSCSVMHEALHNHYTQKSLSLSP | |
| FTL004-h | QVOLVOSGAEVKKPGASVKVSCKASGYTFTDYNVHWIRQAPGOGLEW | 29 |
| u-7 | IGYFYPRNGATHYNQKFTGRATLTVDTSTSTAYMEISSLRSEDTAVYFCA | |
| | RGETPGTFPYWGQGTLVTVSSASTKGPSVFPLAPSSKSTSGGTAALGCL | |
| | VKDYFPEPVTVSWNSGALTSGVHTFPAVLQSSGLYSLSSVVTVPSSSLGT | |
| | QTYICNVNHKPSNTKVDKKVEPKSCDKTHTCPPCPAPELLGGPSVFLFPP | |
| | KPKDTLMISRTPEVTCVVVDVSHEDPEVKFNWYVDGVEVHNAKTKPREEQ | |
| | YNSTYRVVSVLTVLHODWLNGKEYKCKVSNKALPAPIEKTISKAKGQPREPQ | |
| | VYTLPPSRDELTKNQVSLTCLVKGFYPSDIAVEWESNGQPENNYKTTPPVLD | |
| | SDGSFFLYSKLTVDKSRWQQGNVFSCSVMHEALHNHYTQKSLSLSPGK | |
| FTL004-7 | QVOLVOSGAEVKKPGASVKVSCKASGYTFTDYNVHWIRQAPGOGLEW | 31 |
| | IGYFYPRNGATHYNQKFTGRATLTVDTSTSTAYMEISSLRSEDTAVYFCA | |
| | RGETPGTFPYWGQGTLVTVSSASTKGPSVFPLAPSSKSTSGGTAALGCL | |
| | VKDYFPEPVTVSWNSGALTSGVHTFPAVLQSSGLYSLSSVVTVPSSSLGT | |
| | QTYICNVNHKPSNTKVDKKVEPKSCDKTHTCPPCPAPELLGGPDVFLFP | |
| | PKPKDTLMISRTPEVTCVVVDVSHEDPEVKFNWYVDGVEVHNAKTKPREE | |
| | QYNSTYRVVSVLTVLHQDWLNGKEYKCKVSNKALPAPEEKTISKAKGQPREP | |
| | QVYTLPPSREEMTKNQVSLTCLVKGFYPSDIAVEWESNGQPENNYKTTPPVL | |
| | DSDGSFFLYSKLTVDKSRWQQGNVFSCSVMHEALHNHYTQKSLSLSP | |
| | | SEQ ID |
| Antibody | Light chain | NO: # |
| FTL004-h | DIVLTQSPASLAVSPGORATITCRASESVDNFGITFMHWYQQKPGQPPKL | 34 |
| u-1/7 and | LIYRASNLESGVPARFSGSGSRTDFTLTINPVEANDTANYYCOQSSKDPR | |
| FTL004-1/ | TFGQGTKLEIKRTVAAPSVFIFPPSDEQLKSGTASVVCLLNNFYPREAKV | |
| 7 | QWKVDNALQSGNSQESVTEQDSKDSTYSLSSTLTLSKADYEKHKVYAC | |
| | EVTHQGLSSPVTKSFNRGEC | |
| FTL004-h | DIQLTQSPSSLSASVGDRVTITCQASESVDNFGITFMHWYQQKPGKAPK | 35 |
| u-6 and | LLIYRASNLETGVPSRFSGSGSRTDFTFTISSLQPEDIATYYCQQSSKDPR | |
| FTL004-6 | TFGQGTKLEIKRTVAAPSVFIFPPSDEQLKSGTASVVCLLNNFYPREAKV | |
| | QWKVDNALQSGNSQESVTEQDSKDSTYSLSSTLTLSKADYEKHKVYAC | |
| | EVTHQGLSSPVTKSFNRGEC | |

Note:
The variable regions are underlined and the Fc portions are italicized.

Biacore affinity testing was performed on the antibodies before and after Fc engineering, and the results show that the Fc mutation did not cause significant changes in the binding affinities of the antibodies for the antigen. The apoptosis-inducing activity of the antibodies on Romas, Daudi, Raji, and SU-DHL-6 cells was measured before and after Fc mutation using the same method as described above, and the results are shown in Table 14 below. The antibodies, whether before or after Fc mutation, exhibited some apoptosis-inducing activity on the 4 types of CD38-positive cells, and their activity was superior to that of the positive control antibodies Dara and Isa.

TABLE 14
[]The apoptosis-inducing activity of the humanized antibodies
before and after Fc portion engineering
Rate of apoptosis (%)

| Sample | Romas | Daudi | Raji | SU-DHL-6 |
|---|---|---|---|---|
| Dara | 9.78 | 10.62 | 5.73 | 10.39 |
| Isa | 37.91 | 18.23 | 7.99 | 15.94 |
| FTL004-hu-1 | 62.68 | 36.92 | 14.77 | 20.19 |
| FTL004-1 | 64.23 | 40.89 | 7.14 | 21.21 |
| FTL004-hu-6 | 63.62 | 29.31 | 13.84 | 16.84 |
| FTL004-6 | 56.96 | 36.02 | 13.89 | 15.09 |
| FTL004-hu-7 | 31.11 | 34.48 | 12.10 | 19.86 |
| FTL004-7 | 60.27 | 37.35 | 17.77 | 20.31 |

The activity of the antibodies after Fc mutation was measured by ELISA. It can be seen from FIG. 9 that FTL004-1/6/7 are similar in ELISA activity, with the activity of FTL004-1 being slightly higher than that of FTL004-6/7. Compared to FTL004-hu-1/6/7, there was no significant change in activity.

The binding of the antibodies after Fc mutation to Fcγ receptors was measured using methods known in the art. The results are shown in Table 15 below. The results indicate that the antibodies after Fc portion mutation exhibited increased binding to FcγRIIIa (V) and FcγRIIIa (F).

TABLE 15
The binding of the humanized antibodies after Fc portion engineering to Fcγ receptors

| | | Kinetics | | | | |
|---|---|---|---|---|---|---|
| Ligand | Analyte | Chi² (RU²) | ka (1/Ms) | kd (1/s) | KD (M) | Rmax (RU) |
| FcγRIIIa (V176) | FTL004-1 | 1.23E−01 | 7.78E+05 | 2.29E−02 | 2.95E−08 | 3.1 |
| FcγRIIIa (V176) | FTL004-7 | 2.28E−02 | 5.70E+05 | 2.10E−02 | 3.69E−08 | 2.4 |
| FcγRIIIa(F176) | FTL004-1 | 3.07E−01 | 8.63E+09 | 1.68E+02 | 1.95E−08 | 8.0 |
| FcγRIIIa(F176) | FTL004-7 | 6.45E−02 | 5.99E+05 | 1.36E−02 | 2.27E−08 | 4.7 |

### Example 10. In Vivo Pharmacodynamics of Antibodies After Fc Engineering

Animals: 3 NSG mice per group, male; cell line: SU-DHL-6, subcutaneous graft (1×10⁷ cells/0.2 mL/mouse). Dosage and frequency: 10, 30 mg/kg; Q2Wx8; indicators: tumor volume, tumor weight, and the weight of the liver and kidney organs. 10 mg/kg and 30 mg/kg FTL004-1, FTL004-7, and FTL004-hu-1, 30 mg/kg Isa, and FTL004-1+Dara (all 10 mg/kg) were administered by intraperitoneal injection (Q2Wx8).

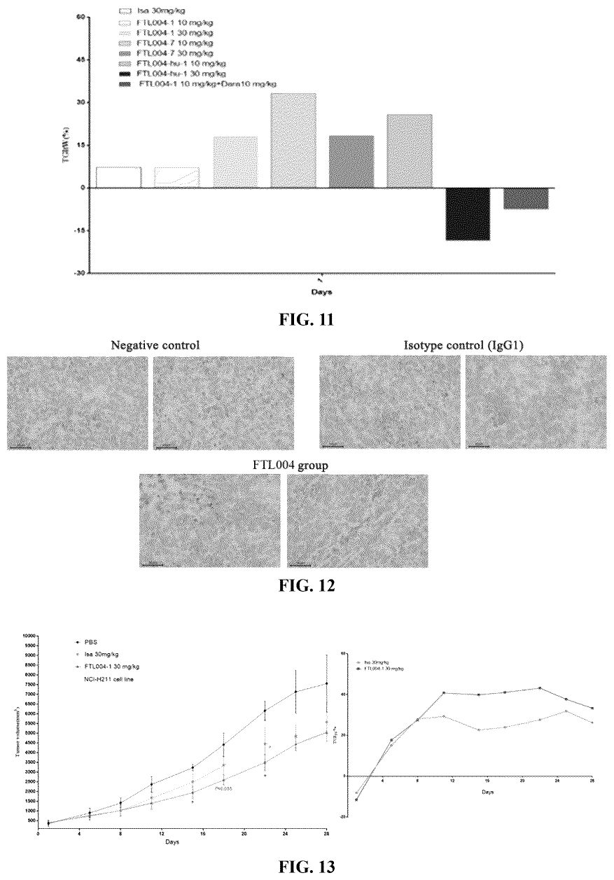

The results are shown in FIG. 10. From the end of the 4th administration to the end of the 8th administration, tumor growth was inhibited in the SU-DHL-6 tumor-bearing mice. The low- and high-dose FTL004-1 groups exhibited dose-dependent responses. At the end of the 29-day dosing experiment, tumors were dissected and weighed, and tumor inhibition rates were calculated (FIG. 11). For the experimental groups, the tumor inhibition rates are as follows: 7.407% for the Isa 30 mg/kg group; 7.407% for the FTL004-1 10 mg/kg group; 18.518% for the FTL004-1 30 mg/kg group; 33.333% for the FTL004-7 10 mg/kg group; 18.518% for the FTL004-7 30 mg/kg group; 25.925% for the FTL004-hu-1 10 mg/kg group; -18.518% for the FTL004-hu-1 30 mg/kg group; and 7.407% for the FTL004-hu-1 10 mg/kg+Dara 10 mg/kg group. The above-described tumor tissues were frozen and sectioned, and positive tissue staining was performed with the antibody FTL004-1. Weak positive reactions were observed with the FTL004 antibodies (FIG. 12).

### Example 11. CD38 Antibody Induces Apoptosis of CD38-Positive Solid Tumor Cells

Through screening of various solid tumor cell lines, small cell lung cancer H211 cells strongly positive for CD38, non-small cell lung cancer A549 cells moderately positive for CD38, non-small cell lung cancer H157 cells weakly positive for CD38, and prostate cancer cell DU145 cells were obtained.

Apoptosis experiments were performed on CD38-positive solid tumor cells. According to Table 16 below, no significant apoptosis was observed on the DU145 and H157 cells with lower CD38 expression abundance in any of the antibody groups; however, on the small cell lung cancer H211 cells that highly express CD38, the different antibodies demonstrated different capacities to induce apoptosis; relatively speaking, the sample FTL004-1 exhibited a superior capacity to induce apoptosis.

TABLE 16
[]The rates of apoptosis of CD38-positive cancer cells induced by
CD38 antibodies

| | Rate of apoptosis (%) | | |
|---|---|---|---|
| Sample name | DU145 | H157 | H211 |
| IgG | 5.31 | 5.77 | 4.75 |
| Dara | 3.68 | 7.93 | 13.24 |
| Isa | 2.34 | 5.36 | 13.57 |
| FTL004-1 | 2.31 | 5.53 | 20.41 |

### Example 12. In Vivo Killing Effect of CD38 Antibodies on Small Cell Lung Cancer H211 Cells

Animals: 4 NCG mice per group, male; cell line: NCI-H211, subcutaneous graft (1×10⁷ cells/0.28 mL/mouse). Dosage and frequency: 30 mg/kg (FTL004-1 and isatuximab); Q2Wx8; indicators: tumor volume, tumor weight, and the weight of the liver and kidney organs.

The experimental results are shown in FIG. 13. It can be seen that 8 consecutive twice-a-week intravenous injections of either 30 mg/kg FTL004-1 or 30 mg/kg Isa tended to inhibit the growth of NCI-H211 subcutaneous tumors, and at the same dose, FTL004-1 inhibited tumors slightly better than Isa.

### Example 13. Binding of CD38 Antibodies to H211 and SU-DHL-6 Cells

By using flow cytometry, the binding of the antibodies Dara, Isa, and FTL004-1 to the CD38-positive cells H211 and SU-DHL-6 cells was measured under different concentration gradients, and their binding EC50 values were determined.

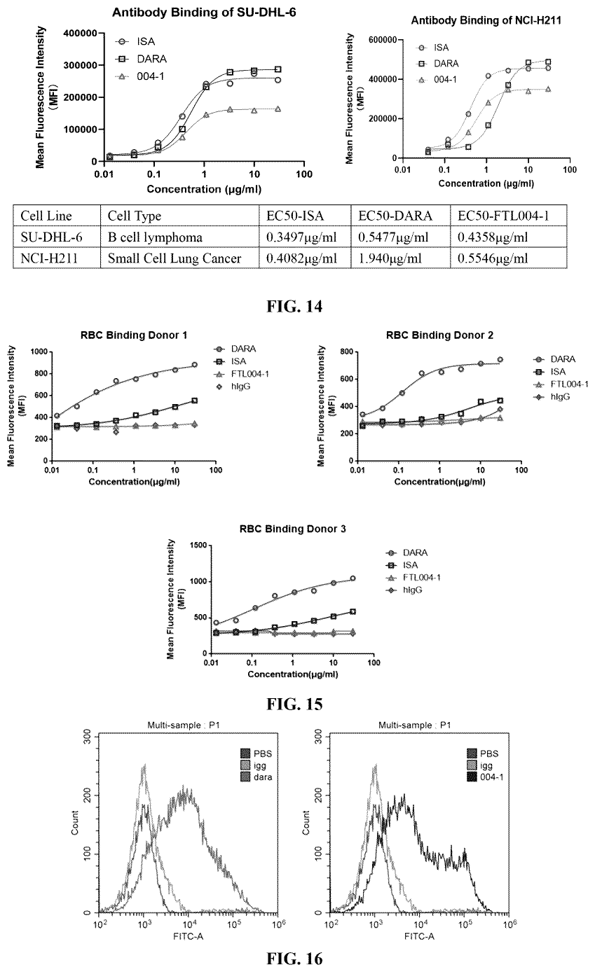

The results show that (FIG. 14): the EC50 value of the antibody FTL004-1 (corresponding to 004-1 in FIG. 14) was similar to that of the antibody Isa, and the value of Dara was relatively high, especially on H211 cells, EC50=1.940 μg/mL.

### Example 14. Binding of CD38 Antibodies to Erythrocytes and PBMCs

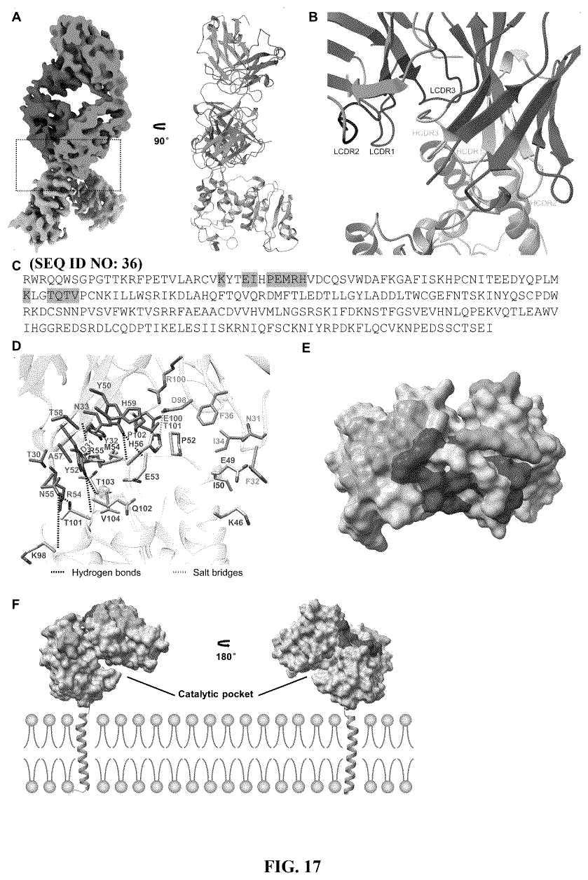

By using flow cytometry, the binding of the CD38 antibodies to rhesus macaque erythrocytes was measured. Dara and Isa did not bind to monkey erythrocytes, and the antibody FTL004-1 exhibited some binding to monkey erythrocytes, with an EC50 value of about 2-5 μg/mL. By using flow cytometry, the binding of the CD38 antibodies to normal human erythrocytes was measured. It can be seen from FIG. 15 that Dara exhibited a relatively high level of binding to healthy (normal) human erythrocytes, and Isa also exhibited some binding to normal human erythrocytes, but the binding was relatively weak; the antibody FTL004-1 did not bind to normal human erythrocytes. By using flow cytometry, the binding of the CD38 antibodies to healthy human PBMCs was measured. It can be seen from FIG. 16 that both Dara and the antibody FTL004-1 (corresponding to 004-1 in FIG. 16) exhibited positive binding reactions with normal human PBMCs.

### Example 15. Three-Dimensional Structural Analysis of CD38/FTL004-Fab Complex

To elucidate the molecular mechanism of FTL004 interaction with human CD38 and identify the binding sites of FTL004 to CD38, we determined the structure of the CD38-ECD-His/FTL004-1 Fab complex using C1 symmetry cryo-electron microscopy (cryo-EM) at a resolution of 3.86 Å.

The human CD38-ECD-His (sequence shown in the table below) was mixed with FTL004-1 Fab fragment at a 1:1 molar ratio and incubated overnight at 4° C. The antibody-antigen mixture was loaded onto a Superdex 200 Increase 10/300 column (Cytiva) equilibrated with SEC buffer (20 mM Tris, 150 mM NaCl, pH 7.2) at a constant flow rate of 0.5 mL/min. The complex peak was collected, concentrated to 0.50 mg/mL in SEC buffer, and immediately used for cryo-EM grid preparation.

TABLE 17
Amino Acid Sequence of CD38 Extracellular Domain

| | |
|---|---|
| RWRQQWSGPGTTKRFPETVLARCVKYTEIHPEMRHVDCQSVWDAFKGAFISKHP | SEQ ID |
| CNITEEDYQPLMKLGTQTVPCNKILLWSRIKDLAHQFTQVQRDMFTLEDTLLGY | NO: 36 |
| LADDLTWCGEFNTSKINYQSCPDWRKDCSNNPVSVFWKTVSRRFAEAACDVVHV | |
| MLNGSRSKIFDKNSTFGSVEVHNLQPEKVQTLEAWVIHGGREDSRDLCQDPTIK | |
| ELESIISKRNIQFSCKNIYRPDKFLQCVKNPEDSSCTSEI | |

The complex structure of CD38/FTL004-1 was determined by cryo-electron microscopy (cryo-EM) at 3.86 Å resolution (FIG. 17A). Five complementarity-determining regions (CDRs) of FTL004-1 were found to participate in CD38 interaction, with LCDR2 being the sole exception (FIG. 17B). FTL004-1 recognizes thirteen residues (K25, E28, 129, P31, E32, M33, R34, H35, K67, T70, Q71, T72, and V73) on human CD38 through hydrogen bonds, salt bridges, and extensive van der Waals interactions (FIGS. 17C and 17D). The CD38/daratumumab (PDB code 7DHA) complex and CD38/isatuximab (PDB code 4CMH) complex were structurally superimposed onto the CD38/FTL004-1-Fab complex (FIG. 17E). This comparative structural analysis revealed that FTL004-1 exhibits a completely distinct binding epitope from daratumumab. Compared to isatuximab's epitope, FTL004-1 has five divergent residues (K25, E28, 129, M33, R34) that may substantially contribute to FTL004-1's enhanced pro-apoptotic activity. FIG. 17F illustrates the putative membrane topology of CD38 and comparative epitope localization of FTL004-1, daratumumab, and isatuximab, demonstrating that FTL004-1's binding site is positioned more proximal to the plasma membrane interface.

It should be understood that although the present invention has been specifically disclosed through preferred embodiments and optional features, one skilled in the art may make modifications, improvements, and variations to the present invention disclosed herein, and these modifications, improvements, and variations are considered to be within the scope of the present invention. The materials, methods, and examples provided herein are representative and exemplary of preferred embodiments and are not intended as limitations on the scope of the present invention.

1. A monoclonal antibody specifically binding to human CD38, or an antigen binding fragment thereof, wherein the monoclonal antibody binds to an extracellular domain of CD38 at amino acid residues comprising at least one or all amino acid residue selected from a group consisting of K25, E28, I29, M33, and R34, wherein the amino acid residue is numbered according to the amino acid sequence of the extracellular domain of CD38 as shown in SEQ ID NO: 36.

2. (canceled)

3. The monoclonal antibody or an antigen binding fragment thereof according to claim 2, wherein the monoclonal antibody or an antigen binding fragment thereof further binds to an extracellular domain of CD38 at amino acid residues comprising at least one or all of P31, E32, H35, K67, T70, Q71, T72 and V73.

4. (canceled)

5. The monoclonal antibody or an antigen binding fragment thereof according to claim 3, wherein the monoclonal antibody or an antigen binding fragment thereof binds to an extracellular domain of CD38 at amino acid residues consisting of K25, E28, I29, P31, E32, M33, R34, H3, K67, T70, Q71, T72 and V73.

6. The monoclonal antibody or an antigen binding fragment thereof according to claim 1, wherein the antigen binding fragment is selected from the group consisting of Fv, scFv, (scFv)₂, Fab, Fab′ and F(ab′)₂.

7. The monoclonal antibody or an antigen binding fragment thereof according to claim 1, wherein the monoclonal antibody or an antigen binding fragment thereof does not bind to healthy human erythrocytes.

8. A pharmaceutical composition, comprising the monoclonal antibody or an antigen binding fragment thereof according to claim 1, and a pharmaceutically acceptable carrier.

9. A method for treatment of a CD38 positive cancer or an autoimmune disease in a subject comprising administering to the subject a therapeutically effective amount of the monoclonal antibody or an antigen binding fragment thereof according to claim 1.

10. The method of claim 9, wherein;
(a) the cancer is a hematologic cancer; preferably the hematologic cancer is selected from multiple myeloma, leukemia, or lymphoma; more preferably the leukemia is selected from acute lymphocytic leukemia (ALL), acute myelogenous leukemia (AML), chronic lymphocytic leukemia (CLL), chronic myelogenous leukemia (CML), juvenile myelomonocytic leukemia (JML), adult T-cell lymphocytic leukemia (ATL), and plasma cell leukemia; and the lymphoma is selected from small lymphocytic lymphoma, lymphoplasmacytic lymphoma, marginal zone lymphoma, follicular lymphoma, mantle cell lymphoma, diffuse large cell B-cell lymphoma, and Burkitt lymphoma; or
(b) the cancer is a solid tumor; preferably the solid tumor is selected from melanoma, lung cancer, squamous non-small cell lung cancer (NSCLC), non-squamous NSCLC, colorectal cancer, prostate cancer, castration-resistant prostate cancer, gastric cancer, ovarian cancer, liver cancer, pancreatic cancer, thyroid cancer, head and neck squamous cell carcinoma, esophagus or gastrointestinal cancer, breast cancer, fallopian tube cancer, brain cancer, urethral cancer, genitourinary cancer, endometrial cancer, cervical cancer, lung adenocarcinoma, renal cell carcinoma (RCC), mesothelioma, nasopharyngeal carcinoma (NPC), esophagus cancer, and gastrointestinal cancer; or
(c) the autoimmune disease is selected from the group consisting of rheumatoid arthritis, systemic lupus erythematosus, multiple sclerosis, myasthenia gravis, type 1 diabetes mellitus, thrombocytopeniarpura, Sjogren's syndrome, Guillain-Barre syndrome, toxic diffuse goiter (Graves' disease), vasculitis, asthma, lymphoma, Alzheimer's disease, amyloid cardiomyopathy, warm autoimmune hemolytic anemia (wAIHA), colorectal carcinoma, immune thrombocytopenia, lupus nephritis, macroglobulinemia, hypergammaglobulinemia, autoimmune hemolytic anemia, and neuromyelitis optica; preferably, the autoimmune disease is systemic lupus erythematosus, warm autoimmune hemolytic anemia, or immune thrombocytopenia.

11-16. (canceled)

17. A method for treatment of an autoimmune disease in a subject comprising administering to the subject a therapeutically effective amount of a monoclonal antibody specifically binding to human CD38, or an antigen binding fragment thereof, wherein the monoclonal antibody comprises three heavy chain complementarity-determining regions HCDR1, HCDR2, and HCDR3, and three light chain complementarity-determining regions LCDR1, LCDR2, and LCDR3, wherein:
(a) the amino acid sequence of the HCDR1 is set forth in SEQ ID NO: 1 or a variant thereof with one to three conservative amino acid substitutions; the amino acid sequence of the HCDR2 is set forth in SEQ ID NO: 2 or a variant thereof with one to three conservative amino acid substitutions; the amino acid sequence of the HCDR3 is set forth in SEQ ID NO: 6 or a variant thereof with one to three conservative amino acid substitutions; the amino acid sequence of the LCDR1 is set forth in SEQ ID NO: 7 or a variant thereof with one to three conservative amino acid substitutions; the amino acid sequence of the LCDR2 is set forth in SEQ ID NO: 9 or a variant thereof with one to three conservative amino acid substitutions; and the amino acid sequence of the LCDR3 is set forth in SEQ ID NO: 11 or a variant thereof with one to three conservative amino acid substitutions;
(b) the amino acid sequence of the HCDR1 is set forth in SEQ ID NO: 1; the amino acid sequence of the HCDR2 is set forth in SEQ ID NO: 2, SEQ ID NO: 3, SEQ ID NO: 4, or SEQ ID NO: 5; the amino acid sequence of the HCDR3 is set forth in SEQ ID NO: 6; the amino acid sequence of the LCDR1 is set forth in SEQ ID NO: 7 or SEQ ID NO: 8; the amino acid sequence of the LCDR2 is set forth in SEQ ID NO: 9 or SEQ ID NO: 10; and the amino acid sequence of the LCDR3 is set forth in SEQ ID NO: 11;
(c) the amino acid sequence of the HCDR1 is set forth in SEQ ID NO: 1; the amino acid sequence of the HCDR2 is set forth in SEQ ID NO: 2; the amino acid sequence of the HCDR3 is set forth in SEQ ID NO: 6; the amino acid sequence of the LCDR 1 is set forth in SEQ ID NO: 7; the amino acid sequence of the LCDR2 is set forth in SEQ ID NO: 9; and the amino acid sequence of the LCDR3 is set forth in SEQ ID NO: 11; or
(d) the amino acid sequence of the HCDR1 is set forth in SEQ ID NO: 1; the amino acid sequence of the HCDR2 is set forth in SEQ ID NO: 2; the amino acid sequence of the HCDR3 is set forth in SEQ ID NO: 6; the amino acid sequence of the LCDR1 is set forth in SEQ ID NO: 8; the amino acid sequence of the LCDR2 is set forth in SEQ ID NO: 10; and the amino acid sequence of the LCDR3 is set forth in SEQ ID NO: 11.

18-20. (canceled)

21. The method of claim 17, wherein the monoclonal antibody comprises a heavy chain variable region (VH) comprising the amino acid sequence set forth in SEQ ID NO: 12 or SEQ ID NO: 14 or a variant having at least 85% sequence identity thereto;
and a light chain variable region (VL) comprising the amino acid sequence set forth in SEQ ID NO: 18 or SEQ ID NO: 21 or a variant having at least 85% sequence identity thereto.

22. The method of claim 21, wherein:
(a) the VH comprises the amino acid sequence set forth in SEQ ID NO: 12 or a variant having at least 85% sequence identity thereto; and the VL comprises the amino acid sequence set forth in SEQ ID NO: 18 or a variant having at least 85% sequence identity thereto;
(b) the VH comprises the amino acid sequence set forth in SEQ ID NO: 12 or a variant having at least 85% sequence identity thereto; and the VL comprises the amino acid sequence set forth in SEQ ID NO: 21 or a variant having at least 85% sequence identity thereto; or
(c) the VH comprises the amino acid sequence set forth in SEQ ID NO: 14 or a variant having at least 85% sequence identity thereto; and the VL comprises the amino acid sequence set forth in SEQ ID NO: 18 or a variant having at least 85% sequence identity thereto.

23. The method of claim 17, wherein an Fc portion of the monoclonal antibody is modified to enhance binding to FcγRIIIa (V) and/or FcγRIIIa (F).

24. The method of claim 23, wherein Fc portion comprises the amino acid sequence set forth in SEQ ID NO: 27 or a variant having at least 85% sequence identity thereto.

25. The method of claim 17, wherein the monoclonal antibody comprises a heavy chain comprising the amino acid sequence set forth in SEQ ID NO: 28, SEQ ID NO: 29, SEQ ID NO: 30, or SEQ ID NO: 31 or a variant having at least 85% sequence identity thereto, and a light chain comprising the amino acid sequence set forth in SEQ ID NO: 34 or SEQ ID NO: 35 or a variant having at least 85% sequence identity thereto.

26. The method of claim 25, wherein
(a) the heavy chain comprises the amino acid sequence set forth in SEQ ID NO: 28 or a variant having at least 85% sequence identity thereto, and the light chain comprises the amino acid sequence set forth in SEQ ID NO: 34 or a variant having at least 85% sequence identity thereto;
(b) the heavy chain comprises the amino acid sequence set forth in SEQ ID NO: 28 or a variant having at least 85% sequence identity thereto, and the light chain comprises the amino acid sequence set forth in SEQ ID NO: 35 or a variant having at least 85% sequence identity thereto;
(c) the heavy chain comprises the amino acid sequence set forth in SEQ ID NO: 29 or a variant having at least 85% sequence identity thereto, and the light chain comprises the amino acid sequence set forth in SEQ ID NO: 34 or a variant having at least 85% sequence identity thereto;
(d) the heavy chain comprises the amino acid sequence set forth in SEQ ID NO: 30 or a variant having at least 85% sequence identity thereto, and the light chain comprises the amino acid sequence set forth in SEQ ID NO: 34 or a variant having at least 85% sequence identity thereto;
(e) the heavy chain comprises the amino acid sequence set forth in SEQ ID NO: 30 or a variant having at least 85% sequence identity thereto, and the light chain comprises the amino acid sequence set forth in SEQ ID NO: 35 or a variant having at least 85% sequence identity thereto; or
(f) the heavy chain comprises the amino acid sequence set forth in SEQ ID NO: 31 or a variant having at least 85% sequence identity thereto, and the light chain comprises the amino acid sequence set forth in SEQ ID NO: 34 or a variant having at least 85% sequence identity thereto.

27. The method of claim 17, wherein the antigen-binding fragment is selected from an Fv, an scFv, an (scFv)₂, a Fab, a Fab′, and a F(ab′)₂.

28. The method of claim 17, wherein the monoclonal antibody:
(a) is of IgG1 type; and/or
(b) does not bind to healthy human erythrocytes; and/or
(c) has a 10⁻⁹ M level affinity constant Kd as determined by Biacore.

29-30. (canceled)

31. The method of claim 17, wherein the autoimmune disease is selected from the group consisting of rheumatoid arthritis, systemic lupus erythematosus, multiple sclerosis, myasthenia gravis, type 1 diabetes mellitus, thrombocytopenia purpura, Sjogren's syndrome, Guillain-Barré syndrome, toxic diffuse goiter (Graves' disease), vasculitis, asthma, lymphoma, Alzheimer's disease, amyloid cardiomyopathy, warm autoimmune hemolytic anemia (wAIHA), colorectal carcinoma, immune thrombocytopenia, lupus nephritis, macroglobulinemia, hypergammaglobulinemia, autoimmune hemolytic anemia, and neuromyelitis optica.

32. The method of claim 31, wherein the autoimmune disease is systemic lupus erythematosus, warm autoimmune hemolytic anemia, or immune thrombocytopenia.
